SEQUENCE LISTING (18 sequences)

SEQ ID NO: 1 - protein, 10 aa, artificial
  TMSC3 limbin target epitope
EDLVEKVRGE

SEQ ID NO: 2 - DNA, 30 bp, artificial
  limbin target epitope
gaagatctggtggaaaaagtgcgcggcgaa

SEQ ID NO: 3 - protein, 259 aa, artificial
  TMSC3
ALTQPSSVSANLGGTVEITCSGGSGSYGWFQQKSPGSAPVTVIYESNKRPSDIPSRFSGSKSGSTGTLTITGVQAEDEAVYFCGSRDSSGSAYAFATGTTLTVLGQSSRSSTVTLDESGGGLQAPGGALSLVCKASGFTFSSYCMQWVRQAPGKGLEFVAGIDDDGSWTAYGAAVKGRATILRDNGQSTVRLQLNNLRAEDTATYYCAKTAGGSYYGCENIDAWGHGTEVIVSSTSGQAGQHHHHHHGAYPYDVPDYAS

SEQ ID NO: 4 - DNA, 777 bp, artificial
  TMSC3
gccctgactcagccgtcctcggtgtcagcaaacctgggaggaaccgtcgagatcacctgctccgggggtagtggcagctacggctggttccagcagaagtctcctggcagtgcccctgtcactgtgatttatgaaagcaacaagagaccctcggacatcccttcacgattctccggttccaaatccggctccacgggcacattaaccatcactggggtccaagccgaggacgaggctgtctatttctgtgggagcagggacagcagcggtagtgcttatgcatttgcgaccgggacaaccctgaccgtcctaggtcagtcctctagatcttccaccgtgacgttggacgagtccgggggcggcctccaggcgcccggaggagcgctcagcctcgtctgcaaggcctccgggttcaccttcagcagttactgcatgcagtgggtgcgacaggcgcccggcaaagggctggagttcgtcgctggtattgatgatgatggtagttggacagcatacggggcggcggtgaagggccgtgccaccatcttgagggacaacgggcagagcacagtgaggctgcagctgaacaacctcagggctgaggacaccgccacctactactgcgccaaaactgctggtggtagttactatggttgtgaaaatatcgacgcatggggccacgggaccgaagtcatcgtctcctccactagtggccaggccggccagcaccatcaccatcaccatggcgcatacccgtacgacgttccggactacgcttct

SEQ ID NO: 5 - protein, 237 aa, artificial
  TMSC1
ALTQPSSVSANLGGTVEITCSGGDIYAGSHYYGWYQQKSPGSAPVTVIYDNTNRPSNIPSRFSGSASGSTNTLTITGVQADDEAVYYCGSIDSTTDVGIFGAGTTLTVLGQSSRSSAVTLDESGGGLQTPGGGLSLVCKASGFSISSYPMEWVRQAPDKGLEFVAGIGGSGSGTKYGVAVKGRATISRDNGQSTVRLQLNNLRAEDTATYYCARSGCYDCAGQIDAWGHGTEVIVSS

SEQ ID NO: 6 - DNA, 711 bp, artificial
  TMSC1
gcgctgacccagccgagcagcgtgagcgcgaacctgggcggcaccgtggaaattacctgcagcggcggcgatatttatgcgggcagccattattatggctggtatcagcagaaaagcccgggcagcgcgccggtgaccgtgatttatgataacaccaaccgcccgagcaacattccgagccgctttagcggcagcgcgagcggcagcaccaacaccctgaccattaccggcgtgcaggcggatgatgaagcggtgtattattgcggcagcattgatagcaccaccgatgtgggcatttttggcgcgggcaccaccctgaccgtgctgggccagagcagccgcagcagcgcggtgaccctggatgaaagcggcggcggcctgcagaccccgggcggcggcctgagcctggtgtgcaaagcgagcggctttagcattagcagctatccgatggaatgggtgcgccaggcgccggataaaggcctggaatttgtggcgggcattggcggcagcggcagcggcaccaaatatggcgtggcggtgaaaggccgcgcgaccattagccgcgataacggccagagcaccgtgcgcctgcagctgaacaacctgcgcgcggaagataccgcgacctattattgcgcgcgcagcggctgctatgattgcgcgggccagattgatgcgtggggccatggcaccgaagtgattgtgagcagc

SEQ ID NO: 7 - protein, 234 aa, artificial
  TMSC2
ALTQPSSVSANPGETVKITCSGSSDYAYGWYQQKSPGSAPVTVIYNNNKRPSDIPSRFSGSKSGSTGTLTITGVQAEDEAVYYCGSVGDMYVGIFGAGTTLTVLGQSSRSSTVTLDESGGGLQTPGGGPSLLCKASGFSLSDYGMHWVRQAPGKGLEYVAGISTDGSWTGYGSAVKGRATISRDNGQSTVRLQLNDLRAEDTGIYFCAKSAAVGGWHSGFIDAWGHGTEVIVSS

SEQ ID NO: 8 - DNA, 702 bp, artificial
  TMSC2
gcgctgacccagccgagcagcgtgagcgcgaacccgggcgaaaccgtgaaaattacctgcagcggcagcagcgattatgcgtatggctggtatcagcagaaaagcccgggcagcgcgccggtgaccgtgatttataacaacaacaaacgcccgagcgatattccgagccgctttagcggcagcaaaagcggcagcaccggcaccctgaccattaccggcgtgcaggcggaagatgaagcggtgtattattgcggcagcgtgggcgatatgtatgtgggcatttttggcgcgggcaccaccctgaccgtgctgggccagagcagccgcagcagcaccgtgaccctggatgaaagcggcggcggcctgcagaccccgggcggcggcccgagcctgctgtgcaaagcgagcggctttagcctgagcgattatggcatgcattgggtgcgccaggcgccgggcaaaggcctggaatatgtggcgggcattagcaccgatggcagctggaccggctatggcagcgcggtgaaaggccgcgcgaccattagccgcgataacggccagagcaccgtgcgcctgcagctgaacgatctgcgcgcggaagataccggcatttatttttgcgcgaaaagcgcggcggtgggcggctggcatagcggctttattgatgcgtggggccatggcaccgaagtgattgtgagcagc

SEQ ID NO: 9 - protein, 229 aa, artificial
  TMSC4
ALTQPSSVSANSGETVEITCSGSGGSYGWFQQKSPGSAPVTLIYDNTNRPSGIPSRFSGSTSGSAGTLTITGVQAEDEAVYFCGGYDSSSNTGIFGAGTTLTVLAVTLDESGGGLQTPGGALSLVCKGSGFTFSSYAMFWVRQAPGKGLEFVAGIDNPGRTPSYGSAVKGRATISRDDWQSTVRLQLSNLRAEDTATYYCTRGDNIYCASGSGGCIDAWGHGTEVIVSS

SEQ ID NO: 10 - DNA, 687 bp, artificial
  TMSC4
gcgctgacccagccgagcagcgtgagcgcgaacagcggcgaaaccgtggaaattacctgcagcggcagcggcggcagctatggctggtttcagcagaaaagcccgggcagcgcgccggtgaccctgatttatgataacaccaaccgcccgagcggcattccgagccgctttagcggcagcaccagcggcagcgcgggcaccctgaccattaccggcgtgcaggcggaagatgaagcggtgtatttttgcggcggctatgatagcagcagcaacaccggcatttttggcgcgggcaccaccctgaccgtgctggcggtgaccctggatgaaagcggcggcggcctgcagaccccgggcggcgcgctgagcctggtgtgcaaaggcagcggctttacctttagcagctatgcgatgttttgggtgcgccaggcgccgggcaaaggcctggaatttgtggcgggcattgataacccgggccgcaccccgagctatggcagcgcggtgaaaggccgcgcgaccattagccgcgatgattggcagagcaccgtgcgcctgcagctgagcaacctgcgcgcggaagataccgcgacctattattgcacccgcggcgataacatttattgcgcgagcggcagcggcggctgcattgatgcgtggggccatggcaccgaagtgattgtgagcagc

SEQ ID NO: 11 - DNA, 39 bp, artificial
  CSCVHo-F
ggtcagtcctctagatcttccgccgtgacgttggacgag

SEQ ID NO: 12 - DNA, 44 bp, artificial
  CSCG-B
ctggccggcctggccactagtggaggagacgatgacttcggtcc

SEQ ID NO: 13 - DNA, 34 bp, artificial
  CSCVK
gtggcccaggcggccctgacctaggacggtcagg

SEQ ID NO: 14 - DNA, 33 bp, artificial
  CKJo-B
ggaagatctagaggactgacctaggacggtcag

SEQ ID NO: 15 - DNA, 42 bp, artificial
  CSC-F
gaggaggaggaggaggaggtggcccaggcggccctgactcag

SEQ ID NO: 16 - DNA, 47 bp, artificial
  CSC-B
gaggaggaggaggaggaggagctggccggcctggccactagtggagg

SEQ ID NO: 17 - DNA, 22 bp, artificial
  ompseq
aagacagctatcgcgattgcag

SEQ ID NO: 18 - DNA, 24 bp, artificial
  gback
gcccccttattagcgtttgccatc

(19) United States
(12) Patent Application Publication
(10) Pub. No.: US 2010/0062038 A1
(43) Pub. Date: Mar. 11, 2010

(54) Markers, Antibodies and Recombinant scFvs for Mesenchymal Stem Cell Sub-populations and Osteoclasts

(75) Inventors: Tyrone Villalard Bowes, Galway (IE); Udo Greiser, Galway City (IE); William James Johnathan Finlay, Dublin (IE); Timothy O'Brien, Galway (IE); Frank Barry, Galway (IE)

Correspondence Address: AXINN, VELTROP & HARKRIDER LLP;Attn. Michael A. Davitz, 114 West 47th Street, New York, NY, 10036, US

(73) Assignee: NATIONAL UNIVERSITY OF IRELAND, GALWAY, Galway (IE)

(21) Appl. No.: 12/529,789

(22) PCT Filed: Mar. 12, 2008
(86) PCT No.: PCT/EP2008/052959

§ 371 Date: Oct. 29, 2009

(30) Foreign Application Priority Data
Mar. 12, 2007 (IE) 2007/0170
Jan. 11, 2008 (IE) 2008/0015

(51) Int. Cl. A61F 2/00; C07K 7/06; C07H 21/04; C07K 16/00; C12N 15/63; G01N 33/566; C12N 5/00; A61K 35/12

(52) U.S. Cl. 424/423; 530/328; 536/23.1; 530/387.9; 435/320.1; 435/7.21; 435/325; 424/93.7; 530/387.3

(57) ABSTRACT

Abstract Markers, antibodies and recombinant scFvs for Mesenchymal Stem Cell sub-populations and osteoclasts. The present invention relates to specific epitopes of surface membrane bound glycoproteins expressed by mesenchymal stem cells and pre-osteoclasts and relates to antibodies such as monoclonal antibodies and recombinant scFv or fragments thereof, raised to the particular epitope and their use in identifying, isolating, and characterization of mesenchymal stem cell sub-populations such as that termed “Stromal Progenitor Cells” (SPCs) in bone marrow and identifying, isolating, and characterization of pre-osteoclasts in peripheral blood. By binding to a specific epitope on the cell surface, limbin/EVC-2 detection and separation by conventional cell sorting methodologies are facilitated.

### FIELD OF THE INVENTION

The present invention relates to specific epitopes of surface membrane bound glycoproteins expressed by mesenchymal stem cells and pre-osteoclasts and relates to antibodies such as monoclonal antibodies and recombinant scFv or fragments thereof, raised to the particular epitope and their use in identifying, isolating, and characterization of mesenchymal stem cell sub-populations such as that termed ‘Stromal Progenitor Cells’ (SPCs) in bone marrow and identifying, isolating, and characterization of pre-osteoclasts in peripheral blood. By binding to a specific epitope on the cell surface, limbin/EVC-2 detection and separation by conventional cell sorting methodologies are facilitated. The invention further relates to a number of other antibodies and scFvs to proteins present on the surface of Mesenchymal stem cell sub-populations and pre-osteoclasts. The present invention also relates to a kit for use of the invention in a one step purification process for stem cell populations found in bone marrow samples (and other adult tissue) and pre-osteoclasts found in peripheral blood samples and the like and to such purification processes.

### BACKGROUND TO THE INVENTION

Adult stem cell therapy holds great promise for damaged tissue repair by means of regeneration of a number of tissue types, including neurological, muscular and cardiovascular tissues and others derived from mesenchymal sources such as cartilage [1-6]. Adult stem cell based therapies raise much less regulatory concerns and adverse public opinions than embryonic stem cells. There is great interest in the areas of regenerative medicine and tissue engineering in the therapeutic potential of non-haematopoietic stem cells obtained from bone marrow.

Mesenchymal stem cells (MSCs) are worthy of particular interest for a number of reasons. The use of cells from the patient's own bone marrow prevents problems with rejection arising and return the damaged tissue to a much healthier state. In addition, the use of MSCs derived from adult human bone marrow or peripheral adult human blood avoids the ethical issues concerning the use of embryological cells for stem cell harvesting. Both MSCs and osteoclasts are believed to be in close co-operation and have been implicated in cancer metastasis and as such provide a target for anti-metastasis cancer therapies [56-58]

Mesenchymal stem cells are pluripotent stem cells that are progenitors of non-haematopoietic tissues. Cultured MSCs retain their potential to differentiate into osteoblasts, chondrocytes, myocytes or adipocytes [1, 3, 7-9]. When cultured without serum in the presence of transformation growth factor (TGF), MSCs differentiate into chondrocytes, while when cultured in serum with ascorbic acid and dexamethasone, they differentiate into osteocytes [10-18]. Such committed cultured cells will differentiate when placed in vivo into damaged tissues (e.g. cartilage)[19]. It has also been observed that MSCs under certain conditions can differentiate into nerve cells [20].

At present, the existing methods for purifying MSCs via the so-called “plating method” involves centrifugation of bone marrow aspirates, re-suspension of cells in cell culture medium and subsequent seeding of the cells into cell culture flasks- or Percoll fractionation of bone marrow aspirates obtained from the iliac crest of donors are crude and enrich for MSCs that are morphologically and phenotypically heterogeneous [1, 3, 7, 9, 21, 22]. It is not possible to identify a stem cell on the basis of its phenotypically visible features. Current Stem cell marker technology is used to identify and enrich for MSCs. All stem cells are coated with stem cell glycoprotein receptors, which act as biological markers, since they have the capability of selectively binding or adhering to tagging molecules e.g. antibodies. There are many types of marker with varying affinities for signalling molecules. Each stem cell type will have a particular combination of receptors, which make the cells distinguishable from each other. Antibodies can bind to the stem cell specific receptors and the antibodies, or combinations of antibodies, which bind, produce a particular profile characteristic of stem cell types. Presently, molecular biological techniques such as fluorescence-activated cell sorting (FACs) are used to isolate and characterize tagged cells. The antigenic profiles of cultured MSCs isolated by either direct plating or current commercially available antibodies are not unique. The cell markers reportedly expressed by cultured MSCs include, CD10, CD13, BMP (bone morphogenetic protein), CD49a, CD61, CD90, CD105, CD106, CD109, CD140b, CD164, CD166, CD172a, but these are all ubiquitous in their expression pattern [2, 3, 7, 9, 22-25]. Cultured mesenchymal stem cells bind the antibodies/express the markers before they commit to differentiation towards lineages. Other assays such as red oil O staining etc. are used, once mesencymal stem cells are fully differentiated. However, many other cell types express many of the markers and so will bind these antibodies. Employing the necessary cocktail of antibodies is an expensive, inefficient and time consuming way of characterising MSCs.

Presently, there are no recombinant scFvs (single chain variable fragments) on the market that will specifically isolate a homogeneous population of MSCs directly from human bone marrow. Only two antibodies have been utilized to characterize MSCs: C15, a single chain antibody, and STRO-1, a mouse IgM antibody, which has been developed using hybridoma technology [26-28]. However, problems associated with C15 and STRO-1 include non-specific binding by flow cytometry, generally resulting from immune tolerance, as a result of poor immunogenicity and lack of memory in the mouse immune response to highly conserved proteins [29-32] The evolutionary relationship between humans and mice is closer than the relationship between humans and chickens. Genetic analysis of genomes revealed that avian and mammal evolutionary divergence occurred earlier than evolutionary divergence of mouse and humans. Consequently, less than optimal antibodies are produced when mice are immunized with a human protein/antigen. A mouse host is more likely to tolerate human sequences because they look “familiar”, due to greater degrees of recognition due to there being less evolutionary distance between the species. A human protein is less recognised in chickens because evolution had more time to raise significant changes in frequently used amino acid stretches. This results in the higher immunogenic response observed in chickens. STRO-1 does not work very well in FACS (fluorescence-activated cell sorting) based assays because the background binding is very high. STRO-1 is not specific to MSCs. In addition, when it does bind to the MSCs, it does not give a good shift of fluorescence intensity. In fact, the resulting signal is just above the background noise of the assay. Presently, there are few other known markers expressed by MSCs in vivo. As a result MSCs are routinely isolated from mammalian bone marrow by FACs sorting using antibodies that are not specific for MSCs and only enrich for cells expressing these markers, e.g. STRO-1 and the LNGFR MACs kit supplied by Miltenyi. These antibodies or combinations of antibodies can be substituted by our one step “TMSC3” characterization method for purifying anMSC sub-population called stromal progenitor cells (SPCs). TMSC1, 2 or 4 offer additional options for MSCs isolation and each of these scFvs can be chosen by investigators based on the specific cell surface markers being recognised.

Alternatively, MSCs may be isolated by way of their tendency to stick to plastic [7]. They readily adhere to plastic in culture and are isolated by prolonged cell culturing which removes the majority non-adherent haematopoietic cell populations, while any adherent terminally differentiated cells gradually die. However, when the resultant sub-populations are examined by light and fluorescence microscopy, they demonstrate a heterogeneous population consisting of fibroblastoid cells, in addition to small round single cells and polygonal cells of different size. Moreover, mutated terminally differentiated cells persisting in culture can give rise to false positive populations [33-35].

Presently, there are no antibody cocktails used to isolate MSCs from bone marrow and the current ‘direct plating’ and commercially available methods for isolation and characterisation of bone marrow derived MSCs do not provide homogenous populations and consist of an assortment of uncommitted and committed progenitors exhibiting divergent stemness.

Current flow cytometric or magnetic cell sorting strategies use a mouse or rat antibody which only enrich MSCs in a limited manner [36-44]. Many of the cell surface markers detected by current commercial antibodies are expressed on a variety of other cells, leading to the selection of unwanted cells, detracting from the purity of the MSCs. In addition, some of the conventionally employed antibodies have undesirable properties e.g., IgMs, which are very difficult to handle, give rise to large backgrounds in the assays and often do not have a high affinity for the target receptors [26, 29, 30]. Presently, there is no commercially available antibody, monoclonal antibody, recombinant scFv or scFV fragment or method that will specifically label and allow purification of homogeneous MSCs directly from human bone marrow. The identification of new cell surface antigens would be of great value in the identification, isolation and further characterisation of MSCs. The present invention provides better characterisation and purification of homogeneous populations of MSCs, and therefore leads to improved therapeutically effective preparations for use in regenerative medicine. Each of the TMSC1, 2, 3 or 4 antibodies offers a one-step purification of sub-populations. The higher specificity of MSC binding produces a higher shift of fluorescence intensity in flow cytometry experiments thus leading to a more sensitive assay. The recombinant scFvs and fragments thereof of the present invention, are superior to conventionally used antibodies, since they exhibit a higher specificity for MSC, by binding through a specific interaction with the Limbin receptor in the case of TMSC3 or corresponding cell markers for TMSC1, 2 and 4.

The future development of highly advanced cell based therapies will largely depend on the ability to obtain and grow the most therapeutically effective cells possible while limiting waste of resources such as material and research hours amplifying contaminated, therapeutically ineffective cells such as those frequently found in conventional MSC preparations.

It is believed that some of the current cell based treatments of diseases e.g. osteoarthritis and cardiovascular disease fail because the effective cells are only a minor fraction of the entire preparation and so are limited in their therapeutic potential. If improved preparations of MSCs could be realised they will lead to significant advances in MSCs based therapies.

United States Publication No: US 2006/0127398 relates to a hybridoma cell line which produces a novel antibody and a related fragment antibody thereof, which is specific to the extracellular I-domain of integrin alpha 10 chain glycoprotein expressed by MSCs which are committed to differentiate into chondrogenic cells. The antibody is used to isolate populations of MSCs, chondrocytes and embryonic stem cells, all of which express integrin alpha 10 beta1.

United States Publication No: US 2006/0088890 discloses a method for the identification and isolation of somatic stem cells by detecting any of defined sequences therein, or angiotensin converting enzyme (ACE), or a fragment thereof, through use of a antibody raised against the antigen of these stem cell markers.

United States Publication No: US 2003/0157078 discloses the expression and use of Osf2 RNA or expressed Osf2 polypeptide as a molecular marker, in the identification and discrimination of primitive pre-mesenchymal, pre-hematopoietic progenitor stem cells from mesenchymal or hematopoietic stem cells. However, none of these antibodies are specific for MSCs.

Limbin is known to be the expression product of the EVC2 (Ellis van Creveld syndrome 2) gene. Techniques of the present invention have, for the first time, determined that limbin is expressed on the cellular surface of mesenchymal stem cells. The invention provides the unexpected result that Limbin can be used to selectively isolate and characterise mesenchymal stem cells. The amino acid sequence and nucleic acid sequence corresponding to the Limbin is deposited under NCBI accession number AY185210.

The current literature suggests a role for Limbin in Ellis von Crefeld syndrome and dwarfism in cattle. Although the exact function of this protein is unknown, it appears to be important for normal growth and development. The EVC2 gene is active in several organs and tissues before birth, including the heart, lungs, liver, kidneys, pancreas, and in muscles used for movement (skeletal muscles). Changes in the EVC2 gene are thought to also cause a skeletal disorder called Weyers acrodental dysostosis. People with this condition can have mild short stature, but often are of average height. Other characteristic features include extra fingers and toes (polydactyl), unusually formed nails, and dental abnormalities. Only one EVC2 mutation has been associated with Weyers acrodental dysostosis. Limbin is also reported expressed by CD14^{+ve} osteoclasts in bone.

### OBJECT OF THE INVENTION

The object of the present invention is to provide improved methods of characterizing and isolating mesenchymal stem cells through the identification of a MSC specific epitope and the provision of antibodies such as monoclonal antibodies, recombinant scFv and scFv fragments capable of binding to the MSC epitope. A further object is to provide a method which is particularly suitable for isolating SPCs.

It is a further object of the present invention to provide a one step, reliable detection kit which can be used to identify and purify meschenymal stem cells, especially SPCs.

It is yet a further object to provide recombinant scFv and scFv fragments that can be used in place of the antibodies, monoclonal antibodies or combinations of antibodies, which are currently used in MSC or SPC research.

It is an object of the present invention to isolate and grow the most therapeutically effective mesenchymal cells whilst minimising waste of laboratory resources such medium, time, cell culture incubator space on amplifying on “contaminating”, therapeutically ineffective cells frequently found in conventional MSC preparations.

It is an object of the present invention to provide antibodies such as monoclonal antibodies, recombinant scFv and scFv fragments, which can be used as a tool in the purification of mesenchymal stem cells and as a means to analyse the cell.

It is yet a further object of the invention to provide methods of treating conditions comprising a degenerative tissue component such as myocardial infarction, osteoarthritis and spinal cord injury.

Such methods entail treating the patient and/or cells for implantation into the patient, with one or more of the cells, vectors, proteins, polypeptides, recombinant scFv, recombinant scFv fragments, and nucleic acid sequences of the invention.

It is a further object of the present invention to provide an epitope that can be used to facilitate targeted drug delivery through coupling of the recombinant scFv to a chemical compound which will bind to limbin epitope and facilitate targeted drug delivery.

### SUMMARY OF THE INVENTION

According to the present invention, there is provided a polypeptide selected from the group comprising an amino acid sequence SEQ ID NO 1: DLVEKVRGE and a sequence substantially homologous to SEQ ID NO 1, which corresponds to the limbin epitope peptide (substantially homologous meaning having at least 70%, at least 80% or at least 90% homology to the said sequences, under stringent conditions).

The invention also relates to a nucleic acid SEQ ID NO: 2 and a sequence substantially homologous to SEQ ID NO: 2, which corresponds to the limbin epitope DNA sequence (substantially homologous meaning having at least 70%, at least 80% or at least 90% homology to the said sequences, under stringent conditions).

The invention provides methods of generating antibodies or antibody fragments against mesenchymal stem cells by use of the polypeptides or nucleotide sequences as described herein.

The invention also provides antibodies such as monoclonal antibodies and recombinant scFvs or fragments of recombinant scFvs raised against any of the group limbin epitopes, limbin polypeptide fragments, and the related SEQ ID NOs and their corresponding homologous sequences herein described (substantially homologous meaning having at least 70%, at least 80% or at least 90% homology to the said sequences, under stringent conditions).

The antibodies, monoclonal antibodies or recombinant scFvs or fragments thereof, of the invention may be raised against any of the group comprising one or more of SEQ ID NO 1 or the polypeptide product of SEQ ID No. 2 and homologous/complementary sequences of the invention, (substantially homologous meaning at least 70%, at least 80% or at least 90% homology, under stringent conditions). The antibodies may be raised by using phage display polypeptide sequences corresponding to the limbin protein/polypeptide or limbin epitope or by way of other conventional techniques.

The present invention relates to other nucleic acids, amino acids and peptide sequences, antibodies and scFvs having a homology of at least 70% under stringent conditions, to the SEQ ID NOs described herein. The homology may be at least 80% or at least 90%.

In a particular embodiment, the invention provides for antibodies such as monoclonal antibodies or recombinant scFvs or scFV fragments thereof, which may be raised against SEQ ID NO: 1 or a substantially homologous polypeptide sequence or to a polypeptide encoded by the nucleic acid sequence of SEQ ID NO: 2 or a substantially homologous/complementary nucleic acid sequences (substantially homologous meaning at least 70%, at least 80% or at least 90% homology, to the said sequences under stringent conditions). Another aspect provides recombinant scFvs or scFV fragments raised against one or more of the limbin epitope or limbin polypeptide fragments, or the related SEQ ID NOs or their substantially homologous/complementary sequences herein described. The antibodies of the invention may be raised against one or more of SEQ ID NO: 1 or a substantially homologous polypeptide sequence or to a polypeptide encoded by the nucleic acid sequence of SEQ ID NO: 2 or a substantially homologous/complementary nucleic acid sequence (substantially homologous meaning at least 70%, at least 80% or at least 90% homology, to the said sequences under stringent conditions). Accordingly, another aspect of the invention provides for antibodies, such as monoclonal antibodies, recombinant scFvs or fragments thereof, comprising polypeptides substantially homologous to the polypeptides selected from the group comprising SEQ ID NO: 3 (which corresponds to the limbin-targeting recombinant scFv TMSC3 peptide), to SEQ ID NO: 5 (which corresponds to the recombinant scFv TMSC1 peptide), SEQ ID NO: 7 (corresponding to the recombinant scFv TMSC2 peptide), and to SEQ ID NO: 9 (which corresponds to the recombinant scFv TMSC4 peptide), substantially homologous meaning at least 70%, at least 80% or at least 90% homology, to the said sequences under stringent conditions.

The invention also provides a nucleic acid encoding an antibody or fragment thereof comprising a sequence substantially complementary to or substantially homologous to a nucleic acid selected from the group comprising SEQ ID NO: 4 (which corresponds to the recombinant scFv TMSC3 DNA sequence), SEQ ID NO: 6 (which corresponds to the recombinant scFv TMSC1 DNA sequence), SEQ ID NO: 8 (which corresponds to the recombinant scFv TMSC2 DNA sequence) and SEQ ID NO: 10 (which corresponds to the recombinant scFv TMSC4 DNA sequence), substantially homologous/complementary meaning at least 70%, at least 80% or at least 90% homology, to the said sequences under stringent conditions.

The antibodies, monoclonal antibodies or recombinant scFvs or fragments thereof, of the invention may be raised against one or more of SEQ ID NO: 1 or a substantially homologous polypeptide sequence or to a polypeptide encoded by the nucleic acid of SEQ ID NO: 2 or to a substantially homologous/complementary sequence thereof.

The antibodies of the invention may be raised against one or more of SEQ ID NO: 1 or a substantially homologous polypeptide sequence or to a polypeptide encoded by the nucleic acid of SEQ ID NO: 2 or to a substantially homologous/complementary sequence thereof, substantially homologous/complementary meaning at least 70%, at least 80% or at least 90% homology, to the said sequences under stringent conditions.

In a particular embodiment, antibodies raised by the methods of the present invention can be selected from the group comprising the sequences characterised by SEQ ID NO: 5, SEQ ID NO: 7 or SEQ ID NO: 9 or SEQ ID NO: 11 or a sequence substantially homologous thereto.

In a further embodiment, the present invention also provides a vector expression system comprising one or more of the nucleic acid molecules of the invention or capable of expressing antibodies such as monoclonal antibodies, recombinant scFvs, recombinant scFv fragments, or polypeptides of the invention. Suitable vector systems include, but are not limited to system such as pcomb3XSS and bacteriophage VCSM13 as expression system and recombinant scFv displaying vectors.

The invention provides a host cell transfected with a nucleic acid selected from the group comprising the nucleic acid sequence of SEQ ID NO: 2, SEQ ID NO: 6, SEQ ID NO: 8, SEQ ID NO: 10 or SEQ ID NO: 12 or a sequence substantially homologous thereto, substantially homologous/complementary meaning at least 70%, at least 80% or at least 90% homology, to the said sequences under stringent conditions.

The host cell is transformed with the genetic material of interest using a vector system as described above.

The invention provides methods of identifying mesenchymal stem cells, including stromal progenitor cells using the antibodies of the present invention. The mesenchymal stem cells expressing the epitopes and/or proteins of the present invention can be identified by the corresponding antibody raised by the methods described herein. The invention provides methods of identifying mesenchymal stem cells including stromal progenitor cells by generating antibodies or antibody fragments against such cells by use of the polypeptides or nucleotide sequences and the vector expression systems and cells as described herein.

The invention provides a method to identify MSCs, comprising using one or more of the cells, vectors, proteins, polypeptides, antibodies such as monoclonal antibodies, recombinant scFvs or recombinant scFv fragments, and nucleic acid sequences of the invention, or combinations thereof. In some aspects of the invention, the identification of MSCs and pre-osteoclasts may comprise the use of an antibody such as a monoclonal antibody, a recombinant scFv or fragment scFv thereof, which has been raised to bind to a stem cell glycoprotein receptor target epitope. The antibodies such as monoclonal antibodies, recombinant scFv or scFv fragments thereof, and nucleic acids in particular may be used as a molecular tag to identify the cell of interest and facilitate separation of the cell using standard molecular biological separation techniques. The tagged cells may be isolated and characterised using cell sorting technologies such as flow cytometry and MACs techniques.

In a particular embodiment, the antibodies of the invention may be employed as diagnostic markers to facilitate purification and characterisation of adult stem cells which express one or more of the proteins of the invention to which the recombinant scFvs and fragments thereof are specific.

Another aspect of the invention provides a method of molecular marking of the cell of interest to monitor the cell during further manipulation and observation particularly of the SPCs and pre-osteoclasts as they mature and in the case of SPCs differentiate into other cell types.

The invention provides methods of identifying SPCs comprising using one or more of the antibodies, polypeptides, and nucleic acid molecules of the invention. The enriched MSCs obtained by the methods of this invention may be cultured in vitro and differentiated into one or more of osteogenic, chondrogenic, adipogenic (methods below). Such cells may be implanted into patients to repair damaged tissue. Under some circumstances, the cells may be re-implanted into the same patients who originally supplied the cells.

The antibodies, such as monoclonal antibodies, recombinant scFvs or scFv fragments thereof have a considerable number of advantageous uses.

(i) to target vectors/gene delivery systems specifically to MSC sub-populations contributing to a new generation of therapeutic agents and genetically modified stem cells. For example, TMSC1, 2, 3 or 4 could be conjugated to viruses, polymers or directly attached to therapeutic DNA molecules. TMSC3-conjugates would deliver genes or drugs specifically to cells that express the target of TMSC3: Limbin/EVC-2.
(ii) a targeting device in gene therapy approaches to genetically modified MSCs for use in regenerative medicine. The recombinant scFv serves as a targeting device because it will specifically detect MSCs and have low background binding to irrelevant cell lines.
(iii) coat prosthetic implants with for example scFv TMSC1, 2, 3 or 4 in order to bind MSCs to sites of injury and aid in the repair of damaged tissue such as cartilage repair in osteoarthritis patients (hip and joint replacements etc.) or cardiovascular disease (e.g. regeneration of damaged heart tissue after infarction).
(iv) coated stents as a treatment option for heart disease (e.g. atherosclerosis treatments and advanced treatments for prevention of in stent restenosis).
(v) coating sutures with for example novel recombinant scFv TMSC1, 2, 3 or 4, which will allow MSCs to stick to the sutures and facilitate improved wound healing.
(vi) to isolate and purify homogenous MSCs from suitable samples. TMSC1, 2, 3, or 4 could be supplied as lyophilised antibody preparations that upon resuspension would be incubated with human bone marrow cells. Targeted MSCs would be bound and detected using an anti-HA tagged secondary antibody and MSCs sorted using conventional FACS SORTERs.
(vii) Due to the evolutionary differences between human and chickens, TMSC1, 2, 3 and 4 may recognise conserved epitopes across mammalian species. Thus TMSC1, 2, 3 and 4 may be used to purify MSCs from other mammalian sources including mouse, rat, rabbits and horses facilitating the investigation of the therapeutic effect of MSCs in various animal models of disease.

In another aspect, the invention provides a kit comprising one or more antibodies or fragments thereof of the invention. The kit may further comprise magnetic beads. The antibodies, recombinant scFvs or fragments thereof may be linked to the magnetic beads/particles. The immobilised scFv beads can be mixed with human bone marrow and targeted MSCs isolated using for example Miltenyi's CliniMACs isolation system. The kit may comprise nucleic acids encoding the antibodies or fragments thereof, or cells transfected so as to express the antibodies thereof, or vectors suitable for transfecting into cells so as to express the antibodies or fragments thereof of the invention.

The invention provides for use of the polypeptides, antibodies, nucleic acids, vector expression system and/or cells of the present invention can be used in the preparation of a medicament for the treatment of a degenerative, cardiovascular, inflammatory or autoimmune disorder. Such disorders include Parkinson's Disease and Alzheimer's Disease, but is not limited to these conditions.

The invention also relates to the use of TMSC1, TMSC2, TMSC3, and TMSC4 in cancer diagnosis

The invention also relates to the humanisation of these antibodies for use in anti-cancer metastasis therapies

In a further aspect the invention provides use of one or more of TMSC1, TMSC2, TMSC3, and TMSC4 as a diagnostic tool for osteoporosis

In fact the compositions and methods of the invention find use in any therapeutic strategy that targets Limbin such as osteoporosis and cancer, and cancer metastasis.

In another aspect, the invention provides for a pharmaceutical composition comprising a therapeutically effective amount of the one or more of the antibodies such as monoclonal antibodies, recombinant scFvs or scFv fragments thereof, MSCs, peptides, nucleic acids, cells or vectors of the invention, alone, or in combination with suitable pharmaceutical buffers and carriers for injection or other administration methods.

In yet another aspect, the invention relates to uses of the antibodies, such as monoclonal antibodies, recombinant scFvs or scFv fragments thereof, of the invention in monitoring the differentiation potential of uncommitted mesenchymal stem cells as they mature. The invention also relates to uses of the novel antibodies, such as monoclonal antibodies, recombinant scFvs or scFv fragments thereof, as a pharmaceutical substance for use as therapeutic agent in the treatment of medical conditions such as heart disease and other conditions. The antibodies, monoclonal antibodies, recombinant scFvs and scFv fragments thereof, may be used as coatings for use on medical devices in the treatment of certain medical conditions. The antibodies, such as monoclonal antibodies, recombinant scFvs or scFv fragments thereof, may also be used as diagnostic markers to facilitate purification, characterisation and enrichment of adult stem cells.

The invention provides for a method of enriching, purifying and/or isolating MSCs comprising the steps of
(i) treating a mixed population of cells with an antibody or antibody fragment (as raised against any of the amino acid/polypeptide sequences described herein, or when generated from any of the nucleic acid sequences herein) and
(ii) isolating those cells identified which react with the antibody as being MSCs.

The invention thus provides an MSC population which is enriched, purified and/or isolated accordingly. Thus, the invention allows for the use of a polypeptide selected from the group comprising SEQ ID NO: 1 or a sequence substantially homologous thereto, SEQ ID NO: 5, SEQ ID NO: 7, SEQ ID NO: 9 and SEQ ID NO: 11 and a sequence substantially homologous thereto to be used for coating a medical device in order to bind MSCs to sites of injury. The invention thus provides a method of preparing such a medical device coated with one or more than one, of said polypeptides. Suitable medical devices include a suture, a tissue scaffold, a bone implant or a wound dressing or the like. In this way, the invention allows such populations to be localised in specific areas to repair damaged tissue. Such a method will allow the treatment of a patient with the isolated MSCs.

The invention allows for use of such a population to identify a further method of isolation, selection and/or enrichment of MSC populations. The purified populations obtained by the methods of the invention could be used as a type of standard or control to check the purity of a second population of MSCs. In this case, the MSCs will be collected in a localised tissue to aid in regeneration and repair of damaged tissue for example. The invention also provides for use of such populations in the manufacture of a pharmaceutical composition comprising a therapeutically effective amount of stems cells, which have been isolated by the methods of the present invention.

The polypeptides of the present invention can be conjugated with a gene delivery system to enable targeted gene therapy to the site of the MSCs. Such a gene delivery system comprises a polypeptide selected from the group comprising SEQ ID NO: 1 or a sequence substantially homologous thereto, SEQ ID NO: 5, SEQ ID NO: 7, SEQ ID NO: 9 or SEQ ID NO: 11 or a sequence substantially homologous thereto.

### EXAMPLE

In one embodiment of the present invention four scFvs were raised and isolated from a cell chicken immune phage display library using conventional methods whereby chickens are immunised with in-vitro cultured human MSCs. The human MSC libraries were created by extracting the mRNA from the spleens and bone marrow from the chickens, producing cDNA library from the genomic mRNA library by recombinant methods, the cDNA was amplified by PCR and subcloned into phagemid expression vectors within regions that promote their expression in E. coli. The phagemid libraries were transformed into E. coli by electroporation and rescued by addition of help phage, the resulting phage expressing scFv. The phage expressing the scFv was added to human MSCs to select for MSC specific binders by three rounds of biopanning. The human MSCs from the final round of panning were harvested and the phage was used to infect E. coli, which were plated and grown to produce the recombinant scFv. Colonies from these plates were picked and analysed for scFv insert using standard PCR techniques. Each PCR reaction was also digested with ALUI to identify clones producing unique scFv sequences. The recombinant scFv obtained in this manner is characterised by SEQ ID NO: 3. The recombinant scFv raised and characterised in this fashion has been denoted scFv (short chain variable fragment) TMSC3. The short chain variable fragment is understood to mean any fragment, which retains the antigen binding specificity of the antibody. One skilled in the art will appreciate that any one of amino acid sequences SEQ ID NOs: 3, 5, 7 or 9, or polypeptides encoded by the nucleic acid sequences SEQ ID NOs: 4, 6, 8 or 10, and those sequences having substantial homology/complementarity to same, can be used to generate scFvs for mesenchymal stem cells.

The present invention provides better characterisation and purification of homogeneous populations of MSCs, and therefore leads to improved therapeutically effective preparations for use in regenerative medicine. The TMSC 1, 2, 3 or 4 method offers a one-step purification of homogeneous MSCs. The higher specificity of MSC binding produces a higher shift of fluorescence intensity in flow cytometry experiments thus leading to a more sensitive assay. TMSC 1-4 finds particular use in identifying SPCs.

The recombinant scFv and fragments thereof of the present invention, are superior to conventionally used antibodies, since they exhibit a specificity for the MSC sub-population known as stromal progenitor cells, by binding through a specific interaction with the limbin receptor.

As the library was raised in chickens, the traditional problem of tolerance associated with generating antibodies in closely related species is greatly overcome [1-2, 6]. A better immune response results from use of chickens, the libraries are easier to create due the lower number of immunoglobulin genes in chickens and the libraries are cheaper and more effective than buying a large commercial library. The TMSC3 scFv from the library of the invention secretes functional scFv into the supernatant for routine purification and analysis by flow cytometry.

An additional advantage of the present invention using TMSC3 over conventional methods concerns the fact that when mouse and rat MAbs are used, it is essential to use fc-blocking agents to prevent non-specific binding of MAb to cells. In the present invention, this is not necessary since chicken derived scFvs are used.

Due to the evolutionary differences between human and chickens, it is anticipated that TMSC3 may recognise a conserved epitope across mammalian species. Thus TMSC3 may be used to purify SPCs from mouse, rat and rabbits facilitating the investigation of the therapeutic effect of SPCs in various animal models of disease. TMSC3 may also be used to purify pre-osteoclasts from mouse, rat and rabbits facilitating the investigation of pre-osteoclasts in various animal models of bone repair and osteoporosis

The novel recombinant scFv may also be used as a targeting device in gene therapy approaches to genetically modify SPCs for use in regenerative medicine and could be used to coat prosthetic implants or sutures in order to bind SPCs to sites of injury and aid in the repair of damaged tissue. Polypeptides, antibodies, monoclonal antibodies, recombinant scFvs or scFV fragments thereof and proteins described herein as being substantially homologous to one or more of polypeptides, peptides, antibodies, fragments thereof and proteins of the present invention are to be understood as also comprising variants, derivatives, and alternatives, particularly those comprising conservative substitutions.

Conservative substitutions may have be defined as substitutions that do not significantly alter either the tertiary structure of the polypeptide or do not significantly alter the activity of the polypeptide, or so not significantly alter the charge or hydrophobicity of the molecule at the target site, or do not significantly alter the bulk or lack thereof of the target site side chain region.

In general, conservative substitutions may be made within each of the following groups: hydrophobic: Met, Gly, Ala, Val, Leu, Ile; long hydrophobic: Leu, Ile; short hydrophobic: Gly, Ala, Val; neutral hydrophilic: Cys, Ser, Thr; hydroxyl group availability: Ser, Thr; acidic: Asp, Glu; basic: Asn, Gln, His, Lys, Arg; positively charged residues: Lys, Arg, His; positively charged, non-cyclic residues: Lys, Arg; residues grossly influencing tertiary structure: Gly, Pro, H is; and aromatic residues: Trp, Tyr, Phe.

The term substantially homologous or substantially complimentary when applied to nucleic acids herein refers to any nucleic acid that is sufficiently homologous to bind to the antisense or sense strand as appropriate at about 37° C. and normal intracellular salinity. The term may also comprise nucleic acids comprising redundant changes in the codon usage.

### BRIEF DESCRIPTION OF THE DRAWINGS

Table 1: Antibodies used in cytometry experiments

FIG. 1: PCR amplification of scFv insert from third round panning clones identifying clones TMSC1, TMSC2, TMSC3 and TMSC4.

FIG. 2: Restriction digest of the TMSC1, 2, 3, and 4 PCR products results in a characteristic fingerprint, a unique pattern of bands that differs from clone to clone.

FIG. 3: Representative purification of TMSC scFvs. (a) TMSC scFv is bound to equilibrated Ni-NTA column and eluted with a step-wise increase in 1M Imidazole concentration. (b) The purity of eluted TMSC3 was confirmed by both silver stain and western blot and produced a band with a molecular weight of approximately 25 KkDa.

FIG. 4: Binding of TMSC scFvs to P0 and P1 hMSC target cells was assessed by flow cytometry using an anti-HA fitc secondary, control cells were incubated with ant-HA fitc alone. TMSC svFv's bound to a sub-proportion of adherent P0 hMSCs (day 8). At P1 (day 15) the proportion of positive cells has either remained relatively constant (TMSC1 and 4) or increased in number (TMSC 2 and 3) indicating that these cells are proliferating. However, subsequent staining of later multiply-passaged cells with lead antibody TMSC3 showed loss of the marker signal.

FIG. 5: Representative binding of TMSC scFvs to human bone marrow cells. Human bone marrow cells were incubated with either TMSC1, 2, 3 or 4 and detected with anti-HA fitc and co stained with either CD3, CD14, CD19, CD34, CD45, CD56, or CD235a for the detection of T cells, monocytes, B cells, HSCs, leucocytes, NK cells and erythrocyte progenitors respectfully. The majority of the TMSC^{+ve} cells are lineage negative and CD45^{+ve}. TMSC1, 2, 3, and 4 bind to the same lineage-ye CD45+ve population of cells at different developmental stages. A very small subset of CD45^{+ve} CD14^{+ve} monocytes also bind TMSC1, 2, 3 and 4. These have been identified as pre-osteoclasts.

FIG. 6: MACs isolation of TMSC target cells from human bone marrow. No colonies were visible with isolations using anti-HA microbeads alone, in contrast CFUs were obtained using each TMSC scFv, pictured 20 days post isolation.

FIG. 7: Morphology, surface phenotype and differentiation potential of TMSC3 MACs isolated cells from three different marrow donors (i, ii, and iii) compared to ‘directly plated’ hMSCs. (a) At P3, TMSC3^{+ve} cells had a typical MSC-like fibroblastic morphology and a CD14^{-ve}, CD45^{-ve}, CD73^{+ve} and CD105^{+ve} FACs profile. Culture expanded TMSC3+ve cells are CD14−ve, indicating that the co-isolated pre-osteoclasts do not proliferate and apoptose in the presence of rapidly proliferating SPCs (b) Representative images indicate positive adipogenesis with deposition of Oil Red O positive vacuoles in cultures treated with adipogenic supplements compared to untreated cultures. Alkaline phosphatase was specifically upregulated in response to osteogenic induction media in both control hMSC and TMSC3 isolated cultures. (c) Differentiation to the chondrogenic phenotype of TMSC3-isolated cells from three different donors compared to a control hMSC culture was determined by the presence of sulfated proteoglycan (GAG) in the extracellular matrix of aggregate cultures exposed to TGF-β3 in serum-free chondrogenic media.

FIG. 8: Representative flow cytometric analysis of TMSC3 demonstrates binding to pre-osteoclasts in human peripheral blood. (a) Whole blood stained with CD14 to identify monocytes. (b) Costaining with MCSF and TMSC3 demonstrated that all TMSC3+ve cells were MCSF+ve. (c) Costaining with CD51/CD61 and TMSC3 demonstrated that all TMSC3+ve cells were CD51/CD61+ve. Pre-osteoclasts are CD14+ve MCSF+ve and CD51/CD61+ve, therefore CD14+ve TMSC3+ve cells in human bone marrow, and blood are pre-osteoclasts.

FIG. 9: Representative binding of TMSC scFvs to mouse bone marrow cells. Mouse bone marrow cells were incubated with either TMSC1, 2, 3 or 4 and detected with anti-HA fitc and co stained with CD44. TMSC scFvs bind to a CD44+ve subset of cells in mouse bone marrow.

FIG. 10: Representative binding of TMSC scFvs to horse bone marrow cells. Horse bone marrow cells were incubated with either TMSC1, 2, 3 or 4 and detected with anti-HA fitc. TMSC scFvs bind to a subset of cells in horse bone marrow.

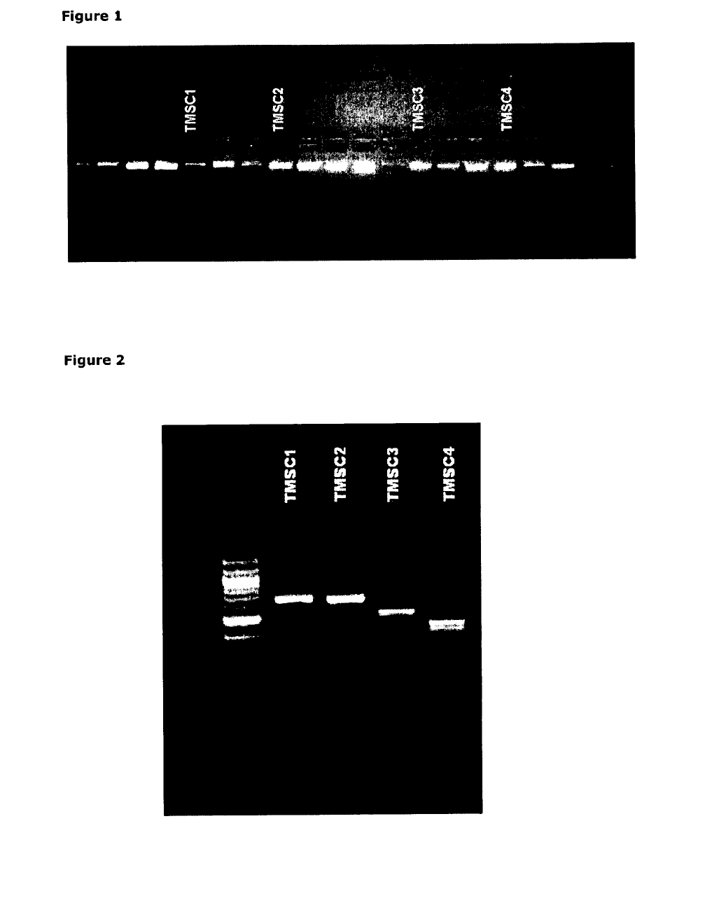

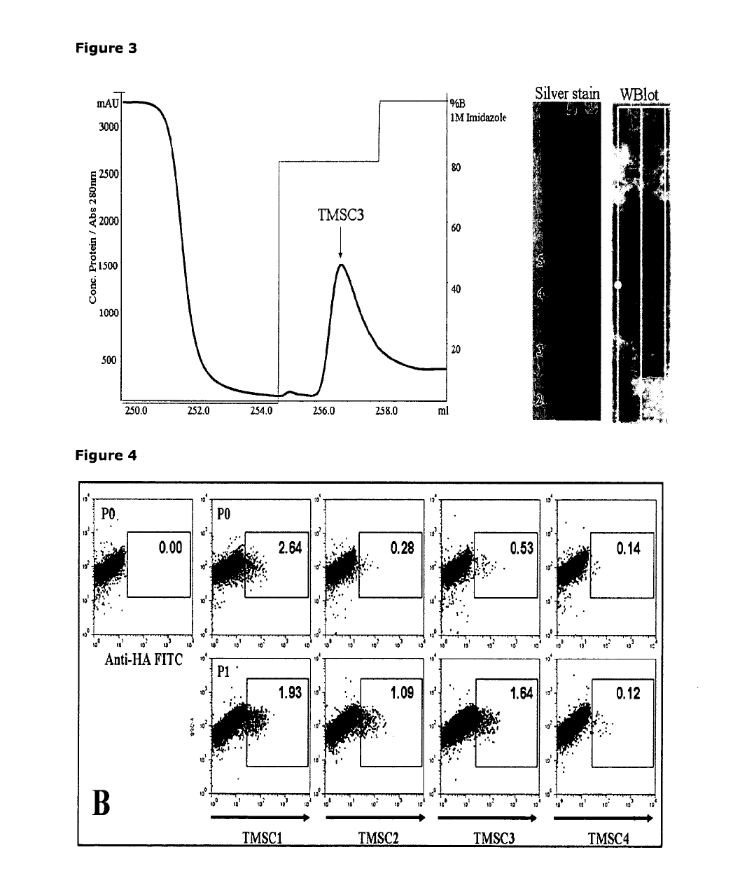

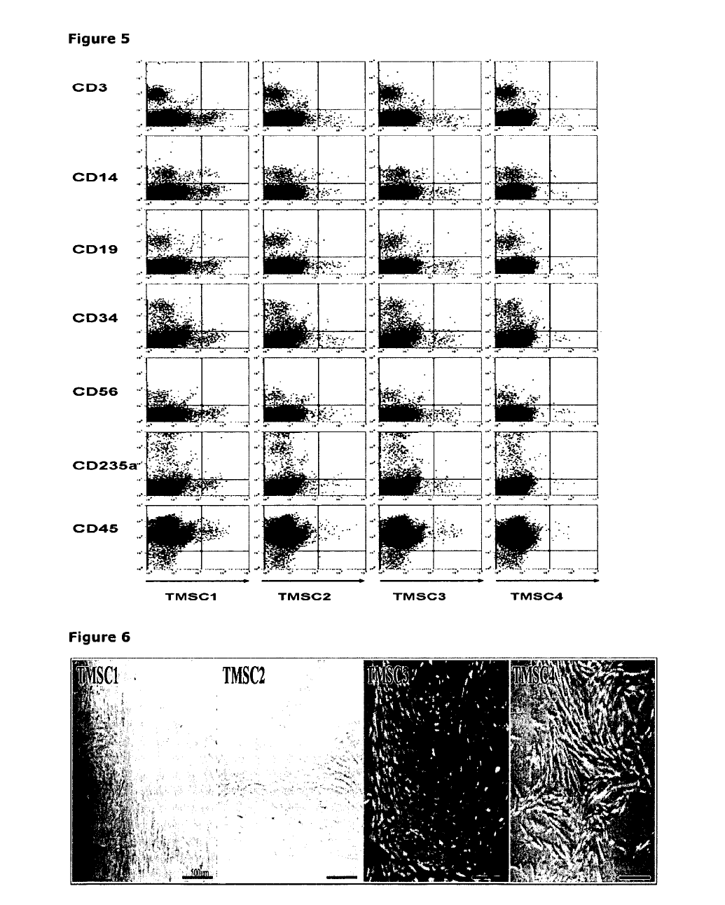

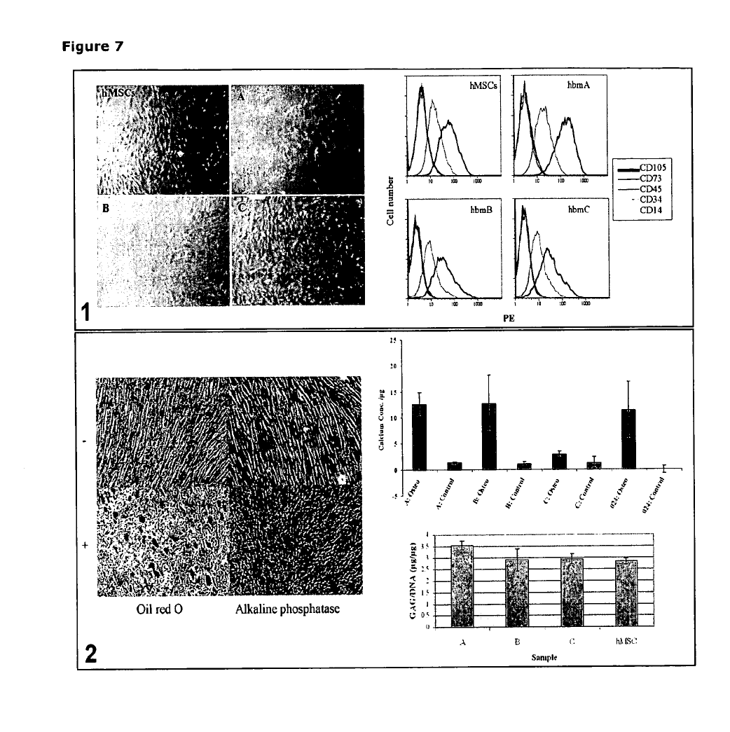

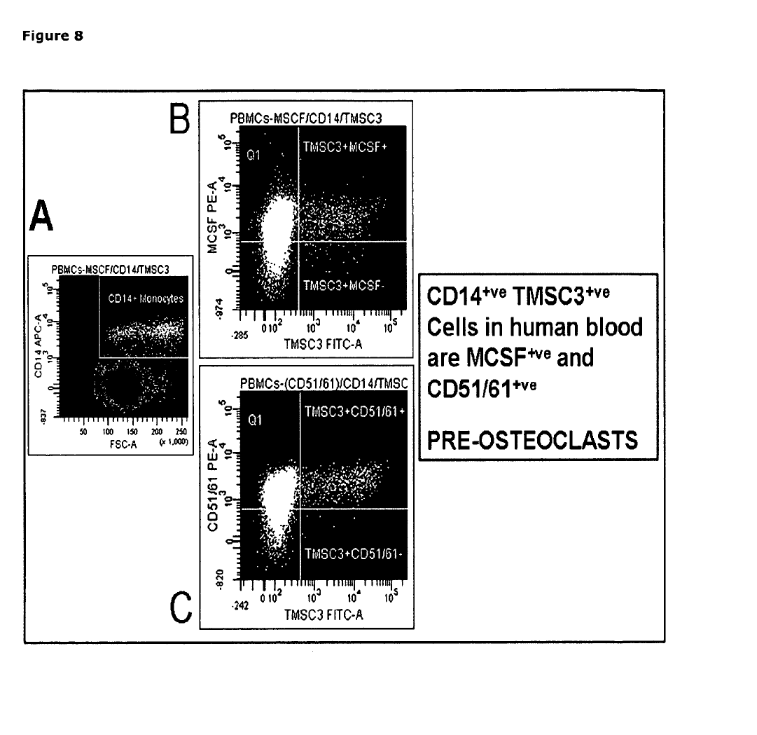

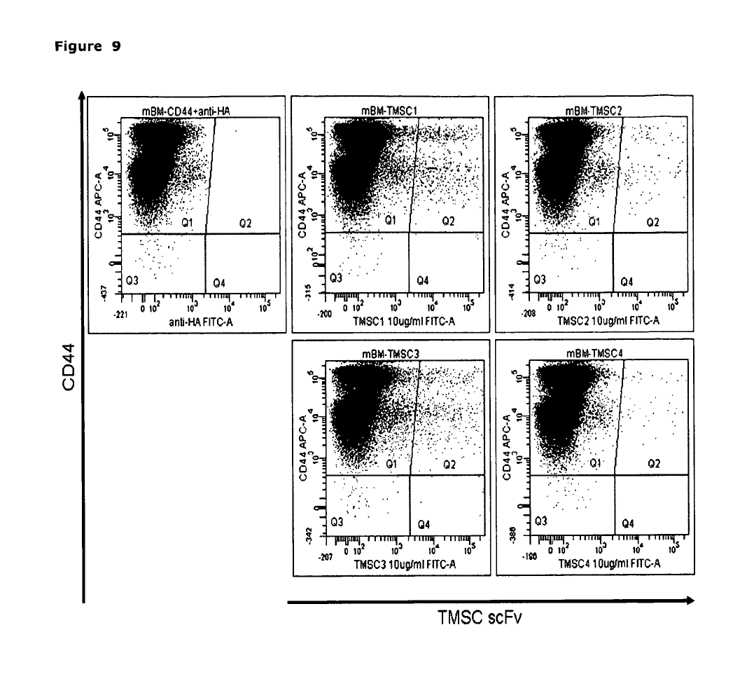

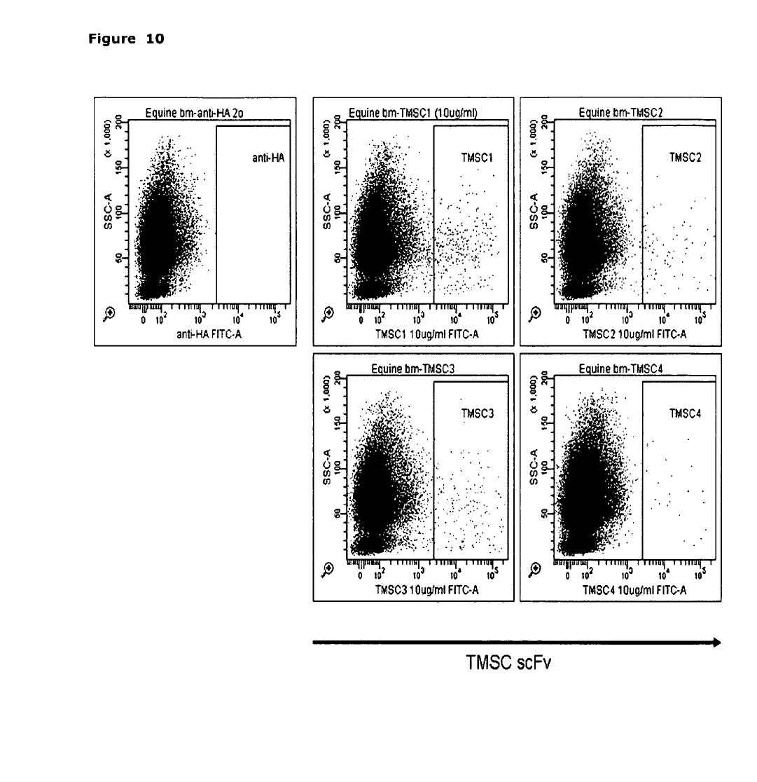

### DETAILED DESCRIPTION OF THE INVENTION

Standard molecular biology and recombinant biotechnology methods were used to create the scFv phage library from spleens and bone marrow of chickens immunised with cultured human mesenchymal stem cells, which were isolated from adult human bone marrow. The scFvs were expressed on the surface of phages and screened for their specificity against cultured human MSCs. Chickens were selected for development of the phage display scFv library since immunisation of chickens with human stem cells gives rise to a substantial and specific immune response. Human, chicken evolutionary separation and lack of tolerance of human antigens by chickens makes chickens an excellent choice for production of an scFv phage display library. A better immune response to highly conserved surface membrane proteins results from use of chickens, the libraries are easier to create due the lower number of immunoglobulin genes in chickens and the libraries are cheaper and more effective than buying a large commercial library.

### Materials

SA brown chickens were obtained from Agro-Bio™ (La Ferte St. Aubin, France); TR1 reagent and SuperScript™ First Strand Synthesis System from Invitrogen™ (Carlsbad, Calif.); Chloroform, 2-propanol, ethanol, agarose and molecular biology grade water from Sigma™ (St. Louis, Mo.); Perfectprep™ Gel Cleanup from Eppendorf™ (Hamburg, Germany); XL I blue and Quick-Pik™ electroelution capsules from Stratagene™ (La Jolla, Calif.); anti IgY and rabbit anti-chicken FITC conjugate from Pierce™ (Rockford, Ill.); RNAse removal solution “Mercury” from CLP direct (San Diego, Calif.); enzyme free cell dissociation solution from Specialty Media™ (Phillipsburg, N.J.) and molecular biology consumables from New England Biolabs™ (Ipswich, Mass.). Electrocompetent XL1 Blue™ (Stratagene™; La Jolla, Calif.); Trypsin, bovine serum albumin, sodium chloride, PEG 8000, glycine, triethylamine, superbroth medium and PBS tablets from Sigma™ (St. Louis, Mo.); Antibiotics from Roche™ (Base1; Switzerland); EBM-2 from Cambrex™ (Berkshire, UK); Micropulser™ (Biorad™; Hercules, Calif.); sequencing primers were obtained from MWG™ (Martinsried, Germany)

### Method for Isolation and Expansion of Human MSCs for Immunisation

Bone marrow aspirates were obtained from the iliac crest of normal donors after informed consent was given. The MSCs were isolated and expanded in culture as described previously in after Percoll fractionation or by direct plating [7, 48].

The aspirates were washed with Dulbecco's phosphate-buffered saline (D-PBS). The cell-containing fraction was gently layered onto a Percoll cushion (1.073 g/ml), at a density of 1−3×10⁸ nucleated cells/25 ml and centrifuged at 1,100×g for 30 min at 20° C. The nucleated cell fraction at the interface with density 1.073 g/ml was collected, washed once with D-PBS and resuspended in MSC culture medium (10% fetal bovine serum (FBS) in Dulbecco's modified Eagle's medium containing 1.0 g/l glucose (DMEM-LG) with antibiotic/antimycotic supplements). Cells were plated at 1.6×10⁵ cells/cm² in T-175 flasks. Cultures were maintained at 37° C. in a humidified atmosphere and 5% CO₂. At the end of P0, adherent colonies were detached by cell scrapping and the cells were cryopreserved in 10% DMSO/90% FBS until used.

### Preparation of Repertoire of Immune scFv Libraries

### Method for Immunization of Chickens

The cryo-preserved stem cells were thawed in a 37° C. water bath and spun down in an Eppendorf™ desktop centrifuge at 1000 rpm for 5 minutes. The freezing medium was aspirated and the cells resuspended in a total of 10 ml of fully supplemented cell culturing medium and left in a 37° C. water bath for 45 minutes. The stem cells were spun down again and washed with PBS solution. The washing step was repeated once. 50,000 cells of each cell line were resuspended in a total volume of 200 μl PBS and injected intradermally under the wing of the chickens. All chickens were pre-bled prior to immunizations. Two chickens (no. 261 and 262) were injected with mesenchymal stem cells, the birds received a second boost injection of 50,000 cells three weeks later to enhance the immune response. A final boost was performed another three weeks later. The chickens were bled on day 32 and the eggs were collected starting three days after the final immunization step. All birds were sacrificed on day 49, spleen and bone marrow was harvested, homogenized and stored in TR1 reagent (Invitrogen™)

Method for Preparation of mRNA from Spleen and Bone Marrow Homogenates

All the equipment used was cleaned with RNAse removal solution (CLP direct) prior to the experiments. Homogenized spleen and bone marrow samples from chickens were thawed in a 30° C. water bath; 10 ml of TR1 reagent was added and mixed thoroughly. The tubes were spun for 10 minutes at 2500 g and 10 ml of each supernatant was transferred into fresh polypropylene centrifugation tubes. 6 ml of chloroform was added under a laminar flow hood and the tubes were inverted several times. After 5 minutes, the samples were mixed again, incubated at room temperature for 5 minutes to allow nuclear proteins to dissociate from the RNA and spun at 4° C., 17,000 g for 15 minutes. The aqueous phase was transferred into fresh tubes, 7 ml of isopropanol added and the RNA was precipitated by centrifugation at room temperature, 15,000 rpm for 20 minutes. The supernatant was discarded, the pellet washed with 70% ethanol and precipitated again by centrifugation at 15,000 rpm for 10 minutes. The purified RNA was dissolved in water and examined for yield and purity in a Shimadzu™ spectrophotometer.

cDNA Synthesis from Chicken mRNA

First-strand cDNA synthesis was carried out according to the instructions in the Invitrogen™ instruction manual included with the SuperScript™ First Strand™ synthesis system for RT-PCR. Briefly, 25 μg of chicken mRNA was mixed in DEPC water with dNTP's, oligo (dT) and incubated at 65° C. for 5 minutes. The samples were chilled on ice for 1 minute, mixed with RT buffer, MgCl₂, DTT (dithiothreitol), RNAse Out™ RNAse inhibitor and incubated at 42° C. for 2 minutes. 5 μl of SuperScript II™ reverse transcriptase was added to the tubes and incubated for 50 minutes at 42° C. All reactions were terminated at 70° C. for 15 minutes, chilled on ice and incubated with RNAse H for 20 minutes at 37° C.

Construction of scFv Libraries

Amplification of the target cDNA using PCR was carried out according to the instructions in given in “Phage display—A laboratory manual” by Carlos Barbas et. al (6). Briefly, short linker scFv libraries were generated for all samples obtained from chickens 261 & 262 (samples were pooled; MSC library), 263 (7 day EPC library), 264 (out-growth library) and 284 (control library; PBS library) using primers CSCVHo-F (sense) (SEQ ID NO 11) and CSCG-B (SEQ ID NO 12) to amplify the chicken V_{H} domains. Similarly, primers CSCVK (sense) (SEQ ID NO 13) and CKJo-B (reverse) (SEQ ID NO 14) were utilized to amplify the V_{λ} domains.

### Primers

CSCVHo-F: chicken V_{H} domain sense: SEQ ID NO: 11
CSCG-B: chicken V_{H} domain antisense: SEQ ID NO: 12
CSCVK: chicken V_{λ} domain sense: SEQ ID NO: 13
CKJo-B: chicken V_{λ} domain antisense: SEQ ID NO: 14

### PCR Round 1

The first-round PCR amplification of chicken V_{H} sequences for the construction of scFv libraries. The primers CSCVHo-F (short linker) are paired with the CSCG-B reverse primer to amplify V_{H} segments from chicken cDNA. The sense primers have a sequence tail that corresponds to the linker sequence that is used in the overlap extension PCR. The reverse primer has a sequence tail containing an Sfi I site; this tail is recognized by the reverse extension primer used in the second round PCR.

0.5 μg of cDNA was amplified in 30 PCR cycles using 60 pmole of each primer according to the following protocol: an initial cycle at temperature of 94° C. for 5 minutes that resulted in the initial denaturation of the cDNA. This was followed by 30 cycles of temperatures 94° C. for 15 seconds; 56° C. for 15 seconds and 72° C. for 90 seconds. A final, one time extension step for 10 minutes at a temperature of 72° C., was included at the end of the PCR protocol. The PCR products were then precipitated with ethanol and sodium acetate, stored at −20° C. for 60 minutes before being spun down at 17,500 g at 4° C., dissolved in water and analysed on a 2% agarose gel. The PCR products were excised from the gel and eluted using Eppendorf Perfectprep™ Gel Cleanup kit.

The amplification of chicken V_{λ} sequences for the construction of scFv libraries.

The CSCVK sense primer is combined with the CKJo-B reverse primer to amplify V_{λ} gene segments from chicken cDNA. CSCVK has a 5′ sequence tail that contains an Sfi I site and is recognized by the sense extension primer in the second round PCR. The reverse primer has a linker sequence tail that is used in the overlap extension.

Briefly, 0.5 μg of cDNA was amplified in 34 PCR cycles using 60 pmole of each primer according to the following protocol: an initial cycle, at temperature of 94° C., for a duration of 4 minutes, that resulted in the initial denaturation of the cDNA. This was followed by 34 cycles of temperatures of 94° C. for 45 seconds; 50° C. for 1 min and 72° C. for 90 sec. A final, one time extension step for 10 minutes at a temperature of 72° C. was included at the end of the PCR protocol. The PCR products were precipitated with ethanol and sodium acetate, stored at −20° C. for 60 minutes, before being spun down at 17,500 g, 4° C., dissolved in water and analysed on a 2% agarose gel. The PCR products were excised from the gel, eluted using Eppendorf's Perfectprep™ Gel Cleanup kit and analysed in a Shimadzu™ spectrophotometer.

### The Overlap Extension PCR

An “overlap extension PCR” was performed to generate full length, short linker single chain antibodies. This PCR combines the chicken V_{H} and V_{λ} fragments for the construction of scFv libraries. The sense and reverse extension primers used in this second round of PCR (CSC-F; SEQ ID NO: 5 and CSC-B; SEQ ID NO: 6) recognize the sequence tails that were generated in the first round of PCR.

Briefly, 100 ng of both V_{H} and V_{λ} PCR products were overlapped and amplified in 25 PCR cycles using 60 pmole of CSC-F and CSC-B primers according to the following protocol: an initial cycle, at temperature of 94° C., for a duration of 5 minutes, that resulted in the initial denaturation of the cDNA. This was followed by 25 cycles of temperatures of 94° C. for 15 seconds; 56° C. for 15 seconds and 72° C. for 2 minutes. A final, one time extension step for 10 minutes at a temperature of 72° C. was included at the end of the PCR protocol. The PCR products were precipitated with ethanol and sodium acetate, stored at −20° C. for 60 minutes, before being spun down at 17,500 g, 4° C., dissolved in water and analysed on a 2% agarose gel. The PCR products were excised from the gel, eluted using Eppendorf's Perfectprep™ Gel Cleanup kit and analysed in a Shimadzu™ spectrophotometer. The PCR products containing the MSC library were digested with Sfi I for 5 hours at 50° C. and subcloned into phagemid vector pCOMB3XSS

### Primers

CSC-F (sense primer): SEQ ID NO: 15
CSC-B (reverse primer): SEQ ID NO: 16
Targeting Repertoire of Immune scFv Libraries to Adult Human Mesenchymal Stem Cells
Transformation of scFv Libraries into E. coli

Electrocompetent XL1 blue bacteria were thawed on ice and mixed with the recombinant scFv libraries in a cuvette on ice. Electroporation was performed with a Micropulser (Biorad™) at 2.5 KV, 25 μF, 200Ω. The bacteria were transferred from the cuvette into glass tubes and incubated at 37° C., 220 rpm for 1 hour.

### Preparation of Phage

Ampicillin (25 μg/ml) and tetracycline (10 μg/ml) were added to the transformed bacteria, incubated another hour at 37° C., 220 rpm and transferred to a 500 ml flask containing 183 superbroth medium, ampicillin and tetracycline. 2 ml of VCSM13 helper phage was added, the samples incubated for 2 h, 37° C., 220 rpm. Kanamycin was added at 25 μg/ml and all cultures were incubated for 6 h, 37° C., 220 rpm. The bacteria were spun down at 3,000 rpm for 15 minutes and the bacterial pellet stored at −80° C. for future plasmid preparation purposes.

The phages were precipitated from the supernatant by addition of 8 g PEG, 6 g NaCl followed by an incubation period of 30 minutes on ice and centrifugation at 15,000 g for 15 minutes at 4° C. The phage pellet was washed once with 1% BSA/PBS and passed through a 0.2μ on filter.

Diversity of scFv Libraries

Aliquots of the original input library and the screened library were electroporated into XL1 blue as described above. The number of transformants was 5×10⁸/library. Twenty clones were randomly picked from a superbroth/carbenicillin plate and the scFv amplified by PCR using primers ompseq (SEQ ID NO 7) and gback (SEQ ID NO 8) The PCR products were digested with Alul for 4 hours at 37° C. and analyzed on 4% gels for unique restriction digest patterns.

### Panning of Phage Libraries on Cultured Mesenchymal Stem Cells

The isolation of the scFvs with reactivity against antigens on the MSCs surface was achieved by iterative cycles of biopanning. Biopanning is typically performed by incubating the library of phage-displayed scFvs with the targets, immobilized either on a plastic plate or on paramagnetic beads. The phages are allowed to bind to the immobilized target on the MSCs, after which the unbound phage is washed away and the bound material is eluted. The eluted phages are then re-amplified and several additional cycles of binding and amplification are performed in order to enrich for phage clones, which have the ability to bind to the desired antigen target.

This MSC-specific phagemid library was transformed into E. coli, and rescued by the addition of VCSM13 helper phage. The resulting phage expressing scFv were added to cultured human MSCs to select for MSC-specific binders in a process known as panning. In total three rounds of panning were performed. In the first round of panning, the phage library was incubated with approximately 2.5×10⁵ cultured human MSCs with the addition of 7×10⁶ PBMCs (to remove non-specific scFv). Cells and phage were then incubated at 4° C. for 30 minutes at 150 rpm. Unbound phage and PBMCs were washed away and phage bound to MSCs were rescued for the next round of panning. Approximately 5×10⁴ human MSCs and 3×10⁶ PBMCs were used in the second round of panning And in the final most stringent round of panning, 5×10³ MSCs and 8×10⁶ PBMCs were used. Human MSCs from the final round of panning were harvested and the phage used to infect E. coli which were subsequently plated and grown overnight at 37° C. Colonies from these plates were picked and analysed for scFv insert using PCR. Each PCR reaction was also digested with ALUI to identify clones producing unique scFv sequences. The recombinant scFvs raised and characterised in this fashion have been denoted TMSC1, TMSC2, TMSC3 and TMSC4 and are characterised by SEQ ID NOs: 5, 7, 3 and 9 respectively. The short chain variable fragment is understood to mean any fragment, which retains the antigen binding specificity of the antibody.

### Primers

### Ompseq: SEQ ID NO:17

### Gback: SEQ ID NO:18

These sequences are primers for library region flanking sequences. Ompseq recognizes a sequence upstream of the scFv in the phagemid pComb3XSS, whilest gback binds to a sequence downstream of the scFv in pComb3XSS. These primers are used to verify that the subcloning into the phagemid has successfully been accomplished (vs. primers 1-6 that were used to amplify chicken antibodies prior to subcloning).

Isolation and Expression of Unique scFv Clones

The unique clones identified from the library of the invention secrete functional scFv into the supernatant for routine purification and analysis by flow cytometry. Frozen glycerol stocks of unique scFv were used to inoculate a Super broth (SB) agar plate containing carbenicillin (carb) and incubated at 37° C. overnight. A single colony from this plate was used to inoculate 2 ml of pre-warmed SB+ carb and incubated at 37° C. for 8 hours. The 2 ml culture was then used to inoculate 250 ml of SB+ carb and incubated for 3 hours at 37° C. at 250 rpm. Expression of scFv was induced by addition of 0.4 ml of 0.5 M IPTG to the culture. After 11 hours the culture was placed on ice and centrifuged at 11,000 rpm for 20 minutes at 4° C. The supernatant was filter sterilised using a 0.2 μm filter. A 6× histidine (HIS) residue is tagged to the scFv to allow protein purification, and the presence of a haemaglutinin (HA) decapeptide tag allows for detection of scFv using anti-HA antibody. Proteins that are engineered to express six histidine residues in tandem can be purified using a resin that contains Ni²⁺ions that are immobilised by covalent linkage to nitrilotriacetic acid (NTA). Immidazole and NaCl were added to the culture supernatant to a final concentration of 1 mM and 0.5M respectfully and was then added to a Ni-NTA agarose column (Qiagen™) and allowed to drip through overnight at 4° C. The column was washed with 5 mM Imidazole and Bound scFv was eluted with 0.25M imidazole, concentrated using centricon filtration system and buffer exchanged with PBS.

Characterization of scFv

The quality of the scFv preparation was analyzed by immunoblotting. A 10% SDS-PAGE was transferred onto a nitrocellulose membrane (30 V, 1 hour). The membrane was blocked over night in 3% BSA/PBS solution and then incubated in a 1:1000 dilution of anti-HA antibody (Roche™) for 1 hour at room temperature. The scFv TMSC3 was detected by addition of a 1:5000 dilution of anti-rat HRP antibody (Roche™) and detected using ECL™ reagents. Silver staining confirmed that the His-tag purification was successful.

Sequencing of scFvs

The scFv clones TMSC1, 2, 3, and 4 were submitted to MWG (Germany) for sequencing analysis using primers ompseq (SEQ ID NO: 17) and gback (SEQ ID NO: 18).

### Cell Surface Target Identification

ScFv TMSC3 was submitted to RZPD™ (Heidelberg) for identification of the cell surface antigen. 50 μg of the scFv was used to detect potential binding domains in human fetal brain cDNA expression library. This library contains 38,000 different proteins. Rabbit anti-HA antibody was used as a secondary antibody.

Limbin is known to be the expression product of the EVC2 (Ellis van Creveld syndrome 2) gene [50]. Techniques of the present invention have, for the fist time, determined that limbin is expressed on the cellular surface of mesenchymal stem cells. The invention provides the unexpected result that limbin can be used to selectively isolate and characterise mesenchymal stem cells.

The current literature suggests a role of limbin in Ellis von Crefeld syndrome and dwarfism in cattle[51]. Although the exact function of this protein is unknown, it appears to be important for normal growth and development [50, 52-55]. Researchers have determined that the EVC2 gene is active in several organs and tissues before birth, including the heart, lungs, liver, kidneys, pancreas, and in muscles used for movement (skeletal muscles) [50, 51]. Changes in the EVC2 gene are thought to also cause a skeletal disorder called Weyers acrodental dysostosis [54]. People with this condition can have mild short stature, but often are of average height. Other characteristic features include extra fingers and toes (polydactyl), unusually formed nails, and dental abnormalities [54]. Only one EVC2 mutation has been associated with Weyers acrodental dysostosis. One skilled in the art will appreciate that limbin protein, can be used to generate scFvs for mesenchymal stem cells.

### FACS Analysis of Human MSCs Using Purified TMSC3

An additional advantage of the present invention using scFvs over conventional methods concerns the fact that when mouse and rat MAbs are used, it is essential to use fc-blocking agents to prevent non-specific binding of MAb to cells. In the present invention, this is not necessary since chicken derived scFvs are used. Cultured MSCs were harvested by cell scrapping and washed with FACs buffer (DMEM media+1% bovine serum albumin (BSA)+0.02% sodium azide). A total of 50 μl of 10 μg/ml of column purified scFv was added to 1×10⁶ cells per sample and incubated for 30 minutes on ice. Cells were washed twice with media+1% BSA and binding of scFv to cells was detected by the addition of 50 μl of a 1:50 dilution of anti-HA FITC antibody for 30 minutes on ice. Samples were washed twice in FACs buffer. Resuspended in 200 μl DMEM+DAPI (200 nM) to gate out dead cells and debris. Flow cytometry analysis was performed on the FACS ARIA with Diva software (BD).

### FACS Analysis of Bone Marrow Using Purified TMSC3.

The ability of TMSC1, 2, 3, and 4 scFvs to bind adult stem cells in human bone marrow was analysed as follows: Human bone marrow was supplied by Cambrex. 1 ml of human bone marrow was centrifuged at 350 g for 5 minutes at 4° C. and resuspended 1 ml of red blood cell lysing buffer and incubated at room temperature for 90 seconds. Cells were centrifuged and washed with FACS buffer (DMEM media+1% BSA+0.02% sodium azide) and incubated with 20% human serum in FACs buffer and incubated on ice for 30 minutes (this is Fc blocking step when using CD markers). The cells were then incubated with 50 μl of purified scFv at 10 μg/ml on ice for 30 minutes. Cells were washed twice with FACs buffer and incubated with 50 μl of a 1:50 dilution of rat anti-HA FITC (Miltenyi) plus 10 μl of either CD3 APC, CD14 APC, CD19 APC Cy7, CD34 APC, CD45 PE Cy7, CD56 APC (BD Biosciences™), and CD235a APC Cy₇ incubated on ice for 30 minutes in the dark. Cells were washed and analysed on a BD FACSARIA sorter.

For mouse bone marrow, mice were sacrificed and bone marrow harvested. Red blood cells were lysed using RBC lysing buffer (Sigma) and cells resuspended in FACs buffer. The cells (1×10⁶ cells) were then incubated with 50 μl of purified scFv at 10 μg/ml on ice for 30 minutes. Cells were washed twice with FACs buffer and incubated with 50 μl of a 1:50 dilution of rat anti-HA FITC (Miltenyi) plus 10 μl of CD44 APC, incubated on ice for 30 minutes in the dark. Cells were washed and analysed on a BD FACSARIA sorter.

Horse bone marrow was treated with red blood cell lysing buffer (BD Pharmlyse) and resuspended in FACs buffer. The cells (1×10⁶ cells) were then incubated with 50 μl of purified scFv at 10 μg/ml on ice for 30 minutes. Cells were washed twice with FACs buffer and incubated with 50 μl of a 1:50 dilution of rat anti-HA FITC (Miltenyi) incubated on ice for 30 minutes in the dark. Cells were washed and analysed on a BD FACSARIA sorter.

Adipogenesis of human MSCs

Human Mesenchymal Stem Cells (hMSCs) in monolayer culture will undergo adipogenic differentiation in the presence of Adipogenic Media containing Dexamethasone, Insulin, 3-Methyl-Isobutylxanthine (MIX) and Indomethacin[45]. Adipogenic differentiation is determined by the formation of lipid vacuoles.

Method: For each assay 2 wells (one'treated' and one ‘control’) of hMSCs are prepared at 2×10⁵ cells per well of a 6 well plate (in a volume of 2-3 mls) and incubated at 37° C. and 5% CO₂. Cells are fed three times per week with hMSC medium until they become confluent. Upon confluency 2.0 ml of the appropriate media is added to each of the wells. On Day 1 (post confluency), treated' wells are fed with filter sterilized Adipogenic Induction Medium (0.2 mL of 1 mM Dexamethasone solution, 0.4 mL of 100 mM Indomethacin solution, 2 ml of Antibiotic-antimycotic solution, 20 ml Fetal Bovine Serum, 2 ml 1 mg/ml Insulin, 0.2 ml of 500 mM MIX, 175.2 ml of HG-DMEM) and ‘control’ wells fed with hMSC growth (20 ml fetal bovine serum, 2 ml Antibiotic-antimycotic solution, 178 ml low glucose DMEM). On Days 5, and 9 ‘treated’ wells are fed with Adipogenic Induction Medium and control wells with hMSC growth medium. While on days 4, 8, 12, 15, and 17 treated wells are fed with Adipogenic Maintenance Medium (2 ml of Antibiotic-antimycotic solution 20 ml Fetal Bovine Serum, 2 ml 1 mg/ml Insulin, 176 ml of HG-DMEM) and control wells with hMSC growth medium. On day 19 the cells are fixed by rinsing each well with 2 ml of sterile DPBS, followed by 10% formalin and incubated for 30 minutes at room temperature, cells are rinsed with 1 ml of DPBS and resuspended in 2 ml of DPBS. Adipogenesis can be quantified using oil red O staining, briefly, pipet working solution of oil red O (Mix 6 parts of stock oil red O (0.3 g/100 ml isopropanol 99%) with 4 parts of distilled water) until the layer of cells is covered and let stand for 5 minutes. Aspirate off and rinse with tap water. Pipet Hematoxylin onto the plate and stain for 1 minute, wash in warm tap water for 4 min and observe staining on a microscope. Stained structures are indicative of the lipid vacuoles which are characteristic of adipogenic cells. Extract the oil red O using isopropanol. Quantify the extracted stain using a spectrophotometer or 96-well plate reader capable of reading absorbance at 490 nm-520 nm

Chondrogenesis Pellet Culture of human MSCs
Method: Cultured MSCs are trypsinized, counted and resuspended in 0.5 ml Complete Chondrogenic Medium (DMEM (high glucose)+6.25 μg/mL bovine insulin, transferring, selenous acid, 5.33 μg/mL linoleic acid, 1.25 mg/mL BSA, 100 mM Dexamethasone, 50 μg/mL Ascorbic acid-2-phosphate, 40 μg/mL Proline, 1 mM Sodium pyruvate, 100 U/mL penicillin, 10,000 μg/mL streptomycin, 250 ng/mL amphotericin B+TGF-β3 @10 ng/mL) such that each chondrogenic pellet contains 2×10⁵ cells in a 15 mL polypropylene conical screw-top tube and incubated at 37° C., 5% CO_{2[}46]. After 24 hours the cells at the bottom of each tube contract into a ball or disc with a diameter of ½ to 1 mm. Medium is changed 3 times a week and Chondrogenic differentiation begins in 1 to 2 weeks. Quadruplicate pellets are set up for each assay time point for day 0, 14, and 21. Two pellets at each time point are used for histological evaluation, and the remaining two are used for biochemical analysis. Chondrogenesis can be quantified. Briefly, pellets are harvested by paraffin embedding or frozen sectioned or placed in the appropriate solution for biochemical analysis (papain digestion of pellets for analysis of sulfated glycosaminoglycans (S-GAG)). For high-quality thin histological sections, harvested pellets are immediately fixed for 30 to 60 min with an isotonic solution of 4% paraformaldehyde or with 10% buffered formalin. Pellets are then transferred from the fixation solution to a 70% ethanol solution in preparation for dehydration, paraffin embedding, sectioning, and staining Export of S-GAG to the extracellular matrix is a hallmark of the chondrogenic phenotype. Determination of S-GAG accumulation depends on the metachromatic change demonstrated by dimethylmethylene blue when complexed to S-GAG, and the consequent shift in the absorption spectrum of the dye. Briefly, pellets are digested with papain, and a solution of DMMB is added to the digest. A positive reaction results in a decrease in absorbance at 595 nm The values obtained are compared against a standard curve prepared using known quantities of chondroitin sulfate.

### Osteogenesis of Human MSCs

Method: Human MSCs plated at a density of 3×10⁴ cells per well in a 6-well plate are grown in the absence and presence of osteogenic supplements (0.1 ml of 1 mM Dex solution, 10 ml of 1M (3-glycerophosphate solution and 5 ml of 10 mM AsAP) in hMSC culture media (100 ml fetal bovine serum, 10 ml Antibiotic-antimycotic solution, 890 ml low glucose DMEM) for 16 days, with media changes performed twice weekly and a media volume of 2 ml per well[47]. Osteogenesis can be quantified by measuring calcium deposition on day 16.

TABLE 1

| Antibody | Cell Type |
|---|---|
| CD3 | T-Cells |
| CD19 | B-Cells |
| CD14 | Monocytes |
| CD56 | Natural Killer Cells |
| CD34 | Hemotopoetic Progenitor Cells |
| CD235a | Glycophorin A/Erythroid progenitors |
| CD45 | Leucocytes |
| CD51/CD61 | Broad expression (osteoclasts) |
| MCSF | Broad expression (osteoclasts) |
| Anti-HA | Detects HA-tagged scFv |

### REFERENCES

1. Baksh, D., L. Song, and R. S. Tuan, Adult mesenchymal stem cells: characterization, differentiation, and application in cell and gene therapy. J Cell Mol Med, 2004. 8(3): p. 301-16.
2. Fukuda, K., Application of mesenchymal stem cells for the regeneration of cardiomyocyte and its use for cell transplantation therapy. Hum Cell, 2003. 16(3): p. 83-94.
3. Kassem, M., M. Kristiansen, and B. M. Abdallah, Mesenchymal stem cells: cell biology and potential use in therapy. Basic Clin Pharmacol Toxicol, 2004. 95(5): p. 209-14.
4. Krugliakov, P. V., et al., [Mesenchymal stem cell transplantation for myocardial reparation of rat experimental heart failure]. Tsitologiia, 2004. 46(12): p. 1043-54.
5. Mizuno, H. and H. Hyakusoku, Mesengenic potential and future clinical perspective of human processed lipoaspirate cells. J Nippon Med Sch, 2003. 70(4): p. 300-6.
6. Turgeman, G., et al., Engineered human mesenchymal stem cells: a novel platform for skeletal cell mediated gene therapy. J Gene Med, 2001. 3(3): p. 240-51.
7. Barry, F. P. and J. M. Murphy, Mesenchymal stem cells: clinical applications and biological characterization. Int J Biochem Cell Biol, 2004. 36(4): p. 568-84.
8. Shandadfar, A., et al., In Vitro Expansion of Human Mesenchymal Stem Cells: Choice of Serum is a Determinant of Cell Proliferation, Differentiation, Gene Expression and Transcriptome Stability. Stem Cells, 2005.
9. Tuan, R. S., G. Boland, and R. Tuli, Adult mesenchymal stem cells and cell-based tissue engineering. Arthritis Res Ther, 2003. 5(1): p. 32-45.
10. Jaiswal, R. K., et al., Adult human mesenchymal stem cell differentiation to the osteogenic or adipogenic lineage is regulated by mitogen-activated protein kinase. J Biol Chem, 2000. 275(13): p. 9645-52.
11. Harris, C. T. and L. F. Cooper, Comparison of bone graft matrices for human mesenchymal stem cell-directed osteogenesis. J Biomed Mater Res A, 2004. 68(4): p. 747-55.
12. Roelen, B. A. and P. Dijke, Controlling mesenchymal stem cell differentiation by TGFBeta family members. J Orthop Sci, 2003. 8(5): p. 740-8.
13. Indrawattana, N., et al., Growth factor combination for chondrogenic induction from human mesenchymal stem cell. Biochem Biophys Res Commun, 2004. 320(3): p. 914-9.
14. Cooper, L. F., et al., Incipient analysis of mesenchymal stem-cell-derived osteogenesis. J Dent Res, 2001. 80(1): p. 314-20.
15. Gaur, T., et al., Canonical WNT signaling promotes osteogenesis by directly stimulating RUNX2 gene expression. J Biol Chem, 2005.
16. Chen, D., M. Zhao, and G. R. Mundy, Bone morphogenetic proteins. Growth Factors, 2004. 22(4): p. 233-41.
17. Mbalaviele, G., et al., Beta-catenin and BMP-2 synergize to promote osteoblast differentiation and new bone formation. J Cell Biochem, 2005. 94(2): p. 403-18.
18. Yang, Y., Wnts and wing: Wnt signaling in vertebrate limb development and musculoskeletal morphogenesis. Birth Defects Res C Embryo Today, 2003. 69(4): p. 305-17.
19. Guo, X. M., et al., [Experimental study of the isolation, culture and in chondrogenic differentiation of human bone mesenchymal stem cell]. Zhonghua Kou Qiang Yi Xue Za Zhi, 2003. 38(1): p. 63-6.
20. Lin, J. R., et al., In vitro culture of human bone marrow mesenchymal stem cell clonies and induced differentiation into neuron-like cells. Di Yi Jun Yi Da Xue Xue Bao, 2003. 23(3): p. 251-3, 264.
21. Ogura, N., et al., Differentiation of the human mesenchymal stem cells derived from bone marrow and enhancement of cell attachment by fibronectin. J Oral Sci, 2004. 46(4): p. 207-13.
22. Zipori, D., Mesenchymal stem cells: harnessing cell plasticity to tissue and organ repair. Blood Cells Mol Dis, 2004. 33(3): p. 211-5.
23. Bacigalupo, A., Mesenchymal stem cells and haematopoietic stem cell transplantation. Best Pract Res Clin Haematol, 2004. 17(3): p. 387-99.
24. Justesen, J., K. Stenderup, and M. S. Kassem, [Mesenchymal stem cells. Potential use in cell and gene therapy of bone loss caused by aging and osteoporosis]. Ugeskr Laeger, 2001. 163(40): p. 5491-5.
25. Luyten, F. P., Mesenchymal stem cells in osteoarthritis. Curr Opin Rheumatol, 2004. 16(5): p. 599-603.
26. Simmons, P. J. and B. Torok-Storb, Identification of stromal cell precursors in human bone marrow by a novel monoclonal antibody, STRO-1. Blood, 1991. 78(1): p. 55-62.
27. Simmons, P. J. and B. Torok-Storb, CD34 expression by stromal precursors in normal human adult bone marrow. Blood, 1991. 78(11): p. 2848-53.
28. Simmons, P. J., et al., Isolation, characterization and functional activity of human marrow stromal progenitors in hemopoiesis. Prog Clin Biol Res, 1994. 389: p. 271-80.
29. Walsh, S., et al., Expression of the developmental markers STRO-1 and alkaline phosphatase in cultures of human marrow stromal cells: regulation by fibroblast growth factor (FGF)-2 and relationship to the expression of FGF receptors 1-4. Bone, 2000. 27(2): p. 185-95.
30. Stewart, K., et al., Further characterization of cells expressing STRO-1 in cultures of adult human bone marrow stromal cells. J Bone Miner Res, 1999. 14(8): p. 1345-56.
31. Gronthos, S, and P J Simmons, The growth factor requirements of STRO-1-positive human bone marrow stromal precursors under serum-deprived conditions in vitro. Blood, 1995. 85(4): p. 929-40.
32. Gronthos, S., et al., The STRO-1+ fraction of adult human bone marrow contains the osteogenic precursors. Blood, 1994. 84(12): p. 4164-73.
33. Sigurjonsson, O. E., K. O. Guethmundsson, and S. Guethmundsson, [Mesenchymal stem cells. A review.]. Laeknabladid, 2001. 87(7/8): p. 627-632.
34. Caplan, A. I., Review: mesenchymal stem cells: cell-based reconstructive therapy in orthopedics. Tissue Eng, 2005. 11(7-8): p. 1198-211.
35. Hui, J. H., et al., Mesenchymal stem cells in musculoskeletal tissue engineering: a review of recent advances in National University of Singapore. Ann Acad Med Singapore, 2005. 34(2): p. 206-12.
36. Gindraux, F., et al., Human and rodent bone marrow mesenchymal stem cells that express primitive stem cell markers can be directly enriched by using the CD49a molecule. Cell Tissue Res, 2006.
37. Heckmann, L., et al., Mesenchymal progenitor cells communicate via alpha and beta integrins with a three-dimensional collagen type I matrix. Cells Tissues Organs, 2006. 182(3-4): p. 143-54.
38. Letchford, J., et al., Isolation of C15: a novel antibody generated by phage display against mesenchymal stem cell-enriched fractions of adult human marrow. J Immunol Methods, 2006. 308(1-2): p. 124-37.
39. Boiret, N., et al., Characterization of nonexpanded mesenchymal progenitor cells from normal adult human bone marrow. Exp Hematol, 2005. 33(2): p. 219-25.
40. Deschaseaux, F., et al., Direct selection of human bone marrow mesenchymal stem cells using an anti-CD49a antibody reveals their CD45med, low phenotype. Br J Haematol, 2003. 122(3): p. 506-17.
41. Jones, E. A., et al., Optimization of a flow cytometry-based protocol for detection and phenotypic characterization of multipotent mesenchymal stromal cells from human bone marrow. Cytometry B Clin Cytom, 2006. 70(6): p. 391-9.
42. Knight, R. L., et al., Tissue engineering of cardiac valves: re-seeding of acellular porcine aortic valve matrices with human mesenchymal progenitor cells. J Heart Valve Dis, 2005. 14(6): p. 806-13.
43. Jones, E. A., et al., Isolation and characterization of bone marrow multipotential mesenchymal progenitor cells. Arthritis Rheum, 2002. 46(12): p. 3349-60.
44. Quirici, N., et al., Isolation of bone marrow mesenchymal stem cells by anti-nerve growth factor receptor antibodies. Exp Hematol, 2002. 30(7): p. 783-91.
45. Rim, J. S., R. L. Mynatt, and B. Gawronska-Kozak, Mesenchymal stem cells from the outer ear: a novel adult stem cell model system for the study of adipogenesis. Faseb J, 2005. 19(9): p. 1205-7.
46. Mehlhorn, A. T., et al., Mesenchymal stem cells maintain TGF-beta-mediated chondrogenic phenotype in alginate bead culture. Tissue Eng, 2006. 12(6): p. 1393-403.
47. Yin, X. X., Z. Q. Chen, and Z. Q. Guo, [Inducing human marrow mesenchymal stem cells into osteoblasts directionally and identification of their osteogenesis characteristics]. Zhongguo Xiu Fu Chong Jian Wai Ke Za Zhi, 2004. 18(2): p. 88-91.
48. Barry, F. P., et al., The monoclonal antibody SH-2, raised against human mesenchymal stem cells, recognizes an epitope on endoglin (CD105). Biochem Biophys Res Commun, 1999. 265(1): p. 134-9.
49. Barbas, C. F., 3rd., et al., Phage Display. A Laboratory Manual. 2001: Cold Spring Harbour Laboratory.
50. Galdzicka, M., et al., A new gene, EVC2, is mutated in Ellis-van Creveld syndrome. Mol Genet Metab, 2002. 77(4): p. 291-5.
51. Takeda, H., et al., Positional cloning of the gene LIMBIN responsible for bovine chondrodysplastic dwarfism. Proc Natl Acad Sci USA, 2002. 99(16): p. 10549-54.
52. Ruiz-Perez, V. L., et al., Mutations in two nonhomologous genes in a head-to-head configuration cause Ellis-van Creveld syndrome. Am J Hum Genet, 2003. 72(3): p. 728-32.
53. van Hagen, J. M., J. A. Baart, and J. J. Gille, [From gene to disease; EVC, EVC2, and Ellis-van Creveld syndrome]. Ned Tijdschr Geneeskd, 2005. 149(17): p. 929-31.
54. Ye, X., et al., A novel heterozygous deletion in the EVC2 gene causes Weyers acrofacial dysostosis. Hum Genet, 2006. 119(1-2): p. 199-205.
55. Tompson, S. W., et al., Sequencing EVC and EVC2 identifies mutations in two-thirds of Ellis-van Creveld syndrome patients. Hum Genet, 2007. 120(5): p. 663-70.
56. Karnoub, A. E., et al. Mesenchymal stem cells within tumour stroma promote breast cancer metastasis. Nature. 449, 557-563 (2007).
57. Goltzman D. Osteolysis and cancer. Journal of Clinical Investigation, 2001. 107(10): p. 1219-20.
58. Atkins, G. J., et al. RANK Expression as a Cell Surface Marker of Human Osteoclast Precursors in Peripheral Blood, Bone Marrow, and Giant Cell Tumors of Bone. Journal of bone and mineral research, 2006. 29(9): p. 1339-49.

1. A polypeptide comprising an amino acid sequence comprising SEQ ID NO: 1 or a sequence substantially homologous thereto.

2. A nucleic acid sequence comprising SEQ ID No. 2 or a sequence substantially homologous thereto.

3. An antibody or fragment thereof raised against a polypeptide as claimed in claim 1 or as encoded by the DNA sequence of claim 2.

4. An antibody comprising the amino-acid sequence SEQ ID No. 3 or the amino-acid sequence encoded by SEQ ID No. 4.

5. A nucleic acid molecule encoding a polypeptide as claimed in any one of claim 1, 3 or 4.

6. A nucleic acid molecule comprising a sequence selected from the group comprising SEQ ID NO:4, SEQ ID NO: 6, SEQ ID NO: 8 or SEQ ID NO: 10 or a sequence substantially homologous thereto.

7. A polypeptide comprising a sequence selected from the group comprising SEQ ID No. 3, SEQ ID NO. 5, SEQ ID NO. 7, SEQ ID NO. 9 or a sequence substantially homologous thereto.

8. A vector expression system comprising the nucleic acid molecule of any one of claim 2, 5 or 6.

9. A cell transfected with a nucleic acid as claimed in any one of claim 2, 5 or 6 or comprising the vector system of claim 8.

10. Use of a polypeptide as claimed in claim 1 or a nucleotide sequence as claimed in claim 2 to generate antibodies or antibody fragments against mesenchymal stem cells or osteoclasts.

11. Use of a polypeptide as claimed in claim 1 or 7, an antibody as claimed in claim 3 or 4, a nucleic acid as claimed in any one of claim 2, 5 or 6, a vector expression system as claimed in claim 8 or a cell as claimed in claim 9, to identify mesenchymal stem cells or osteoclasts.

12. Use as claimed in claim 11 wherein the cell population is an SPC population.

13. Use of a polypeptide as claimed in claim 1 or 7, an antibody as claimed in claim 3 or 4, a nucleic acid as claimed in any one of claim 2, 5 or 6, a vector expression system as claimed in claim 8 or a cell as claimed in claim 9, in the preparation of a medicament for the treatment of a degenerative, cardiovascular, inflammatory or autoimmune disorder.

14. The use as claimed in claim 13, wherein the degenerative, cardiovascular, inflammatory or autoimmune disorder is selected from the group comprising Parkinson's Disease or Alzheimer's disease.

15. A method of enriching, purifying or isolating MSCs from adult tissue or pre-osteoclasts from blood and comprising the steps
(i) treating a mixed population of cells with an antibody or antibody fragment as claimed in claim 3 or when produced by a use as claimed in claim 9; and
(ii) isolating those cells identified which react with the antibody as being MSCs or osteoclasts.

16. A method as claimed in claim 15 wherein the MSCs are SPCs.

17. An MSC population enriched, purified or isolated according to the method of claim 15 or 16.

18. Use of an MSC population as claimed in claim 17 to identify a further method of isolation, selection or enrichment of MSC populations.

19. A method of repairing damaged tissue comprising treating the patient with cells isolated by method of claim 15 or 16 or the population of claim 17.

20. A pharmaceutical composition comprising a therapeutically effective amount of stem cells isolated by method of claim 15 or 16 or as claimed in claim 17.

21. Use of a polypeptide as claimed in claim 1 or an antibody as claimed in any of claim 3, 4 or 7 conjugated with a gene delivery system to enable targeted gene therapy to the site of MSCs

22. A gene delivery system comprising a polypeptide as claimed in claim 1 or an antibody as claim in claim 3, 4 or 7.

23. Use of a polypeptide as claimed in claim 1 or an antibody as claim in claim 3, 4 or 7 for coating a medical device in order to bind MSCs to sites of injury.

24. A medical device coated with a polypeptide as claimed in claim 1 or an antibody as claim in claim 3, 4 or 7.

25. A medical device as claimed in claim 24 selected from a suture, a stent, a tissue scaffold, a bone implant, or a wound dressing.

26. A nucleic acid sequence selected from the group consisting of SEQ ID NOs. 11 to 18.

27. Use of any of SEQ ID Nos 1-18 in a method of cancer diagnosis.

28. A humanised antibody which is a humanised version of SEQ ID NO. 3, 5, 7 or 9.

29. Use of any of SEQ ID Nos 1-18 in a diagnostic method for osteoporosis.

30. A polypeptide, nucleic acid molecule, antibody, vector expression system, cell or cell population, pharmaceutical composition, use, medical device, gene delivery system or method substantially as herein described with reference to the accompanying figures, tables, sequences and appendices.
